(19) United States
(12) Patent Application Publication
(10) Pub. No.: US 2024/0226136 A1
(43) Pub. Date: Jul. 11, 2024

(54) A COMPOSITION FOR IMPROVING, PREVENTING AND/OR TREATING MUSCULAR DYSTROPHY, MULTIPLE SCLEROSIS AND/OR RELATED DISEASES

(71) Applicants: GN Corporation Co Ltd, Yamanashi (JP); Sophy Inc., Kochi (JP)

(72) Inventors: TAKASHI ONAKA, Okayama (JP); Samuel JK ABRAHAM, Yamanashi (JP)

(21) Appl. No.: 18/572,416

(22) PCT Filed: Nov. 7, 2022
(86) PCT No.: PCT/JP2022/041412

§ 371 Date: Dec. 20, 2023

(30) Foreign Application Priority Data
Dec. 10, 2021 (JP) 2021-200800
Mar. 17, 2022 (JP) 2022-042559

(51) Int. Cl. A61K 31/716; A61K 36/062; A61P 21/00

(52) U.S. Cl. CPC A61K 31/716; A61K 36/062; A61P 21/00

(57) ABSTRACT

A beta-glucan, a composition comprising the beta-glucan, and a method of using the beta-glucan for improving, preventing and/or treating muscular dystrophy are provided.

### CROSS-REFERENCE TO RELATED APPLICATIONS

This application claims the benefit of the filing dates of Japanese Application No. 2021-200800, entitled “A composition for improving, preventing and/or treating muscular dystrophy, multiple sclerosis and/or related diseases,” filed on Dec. 10, 2021; and Japanese Application No. 2022-42559, entitled “A composition for improving, preventing and/or treating muscular dystrophy, multiple sclerosis and/or related diseases,” filed on Mar. 17, 2022; the contents of which are incorporated herein by reference in their entirety.

### BACKGROUND

### Field of the Invention

Disclosed is disease-modifying immune-modulatory effects of the N-163 strain of Aureobasidium pullulans-produced 1,3-1,6 Beta glucans in young boys with Duchenne muscular dystrophy, with results of an open-label, prospective, randomized, comparative, multiple-arm clinical study.

### Description of the Related Art

The present invention relates to Beta-glucan for improving, preventing and/or treating muscular dystrophy, multiple sclerosis and/or related diseases. The present invention also relates to a composition comprising said beta-glucan for improving, preventing and/or treating muscular dystrophy, multiple sclerosis and/or related diseases, and to a method of use of Beta-glucan for improving, preventing and/or treating muscular dystrophy, multiple sclerosis and/or related diseases.

### INTRODUCTION

Duchenne muscular dystrophy (DMD) is a devastating X-linked neuromuscular disorder causing severe and progressive weakness of skeletal muscles, leading to loss of ambulation along with concomitant impairment of cardiac and respiratory muscles and early mortality. Mutations in the dystrophin gene, which cause total loss of the dystrophin protein [2], remain the major underlying mechanism. Loss of dystrophin leads to damage of the myofibres' plasma membranes and distorts the structural stability of the plasma, leading to weakness in the myofibres. The weakened myofibres cannot withstand the contraction and relaxation cycles occurring during muscle function. The damage to the membrane releases the cytoplasmic contents, triggering the immune system and causing further muscle fibre damage, weakness and ultimately death [3]. A chronic proinflammatory state ensues, with neutrophil infiltration and macrophages' phagocytosis of the degenerated tissue [3], preventing repair of the muscle damage, which otherwise occurs in a highly orchestrated manner for faster repair in other physiological conditions. The muscle is relatively immunologically privileged, with a low capacity to generate localized immune responses and thus having low rates of abscess and granuloma formation [3]. Therefore, it becomes essential to modulate the inflammation and immunity to resolve the chronic inflammatory state in therapeutic approaches to DMD. Steroid therapy is the most commonly employed immunomodulatory treatment approach. However, side effects include weight gain, weak bones, high blood pressure and behaviour changes in addition to muscle weakness and atrophy in the long term, which contributes to worsening of the disease [4,5]. Thus, there arises the need to develop strategies that will assist in immunomodulation with lesser side effects. Nutritional supplements are a potential option. Beside beta glucans yielding locomotor improvement in zebrafish models of DMD [6], a 1-3,1-6 beta glucan from the N-163 strain of the black yeast Aureobasidium pullulans has been reported to mitigate inflammation, evident by decreases in anti-inflammatory markers such as CD11b, serum ferritin, galectin-3 and fibrinogen. It also produces beneficial immuno-modulation via a decrease in the neutrophil-to-lymphocyte ratio (NLR) and an increase in the lymphocyte-to-CRP ratio (LCR) and leukocyte-to-CRP ratio (LeCR) in human healthy volunteers [7]. Mitigation of lipotoxicity-associated inflammatory cascades in a mouse study has also been reported [8]. Another study done in an animal model of non-alcoholic steatohepatitis (NASH) showed a decrease in liver inflammation and accumulation of F4/80+ cells (macrophages associated with inflammation) [9] in the liver. The present pilot study is to evaluate the immunomodulatory efficacy of the N-163 strain of A. pullulans-produced beta 1-3,1-,6 glucan in comparison with a conventional therapeutic regimen in patients with DMD.

### CITATION LIST

### Patent Literature

[PTL 1] US20090022799A1

### Non-Patent Literature

[NPL-1] Ikewaki N, Kurosawa G, Iwasaki M, Preethy S, Dedeepiya V D, Vaddi S, Senthilkumar R, Levy G A, Abraham S J K. Hepatoprotective effects of Aureobasidium pullulans derived Beta 1,3-1,6 biological response modifier glucans in a STAM-animal model of non-alcoholic steatohepatitis. Journal of Clinical and Experimental Hepatology-2022. https://doi.org/10.1016/j.jceh.2022.06.008
[NPL2] Preethy S, Ikewaki N, Levy G A, Raghavan K, Dedeepiya V D, Yamamoto N, Srinivasan S, Ranganathan N, Iwasaki M, Senthilkumar R, Abraham S J K. Two unique biological response-modifier glucans beneficially regulating gut microbiota and faecal metabolome in a non-alcoholic steatohepatitis animal model, with potential for applications in human health and disease. BMJ Open Gastroenterology 2022; 9:e000985. doi: 10.1136/bmjgast-2022-000985
[NPL3] Raghavan K, Dedeepiya V D, Srinivasan S, Pushkala S, Subramanian S, Ikewaki N, Iwasaki M, Senthilkumar R, Preethy S, Abraham S. Disease-modifying immune-modulatory effects of the N-163 strain of Aureobasidium pullulans-produced 1,3-1,6 Beta glucans in young boys with Duchenne muscular dystrophy: Results of an open-label, prospective, randomized, comparative clinical study. medRxiv 2021.12.13.21267706; doi: 10.1101/2021.12.13.21267706
[NPL4] Vetvicka V, Vetvickova J. Combination Therapy with Glucan and Coenzyme Q10 in Murine Experimental Autoimmune Disease and Cancer. Anticancer Res. 2018 June; 38(6):3291-3297. doi: 10.21873/anticanres.

### SUMMARY OF THE INVENTION

The present invention relates to the following:
(1) A composition for improving, preventing and/or treating muscular dystrophy and/or multiple sclerosis, comprising a beta-glucan.
(2) The composition of (1), in which the beta-glucan comprises a beta-glucan produced by Aureobasidium pullulans N-163 (NITE P-03377).
(3) The composition of (1) or (2), which is used to improve, prevent and/or treat Duchenne muscular dystrophy (DMD).

### BRIEF DESCRIPTION OF DRAWINGS

FIG. 1: CONSORT flow diagram of the trial.

FIG. 2: IL-6 showed the most significant decrease in the N-163 Steroid −ve group compared to other groups. (*p-value significance <0.05).

FIG. 3: Levels of IL-13 levels showed statistically significant increase in control groups and decrease in treatment groups (*p-value significance <0.05).

FIG. 4: Levels of TGF-β showed significant decrease in the N-163 Steroid −ve group compared to other groups (*p-value significance <0.05).

FIG. 5: Levels of dystrophin showed significant increase in the N-163 Steroid −ve group compared to other groups (*p-value significance <0.05).

FIG. 6: Levels of A. haptoglobin; B. CK and C. urine myoglobin in various groups of the study (*p-value significance <0.05).

FIG. 6: Levels of A. haptoglobin; B. CK and C. urine myoglobin in various groups of the study (*p-value significance <0.05).

FIG. 6: Levels of A. haptoglobin; B. CK and C. urine myoglobin in various groups of the study (*p-value significance <0.05).

FIG. 7: Levels of A. titin, B. TNF-α and C. cystatin C in various groups of the study (*p-value significance <0.05).

FIG. 7: Levels of A. titin, B. TNF-α and C. cystatin C in various groups of the study (*p-value significance <0.05).

FIG. 7: Levels of A. titin, B. TNF-α and C. cystatin C in various groups of the study (*p-value significance <0.05).

FIG. 8: 6MWT and NSAA results in various groups of the study (*p-value significance <0.05).

FIG. 8: 6MWT and NSAA results in various groups of the study (*p-value significance <0.05).

FIG. 9 Illustration of vascular smooth muscle dystrophin centred approach to manage muscular dystrophy

FIG. 10A shows quadriceps weight.

FIG. 10B shows gastrocnemius weight.

FIG. 10C shows extensor digitorum longus weight.

FIG. 11A shows plasma ALT.

FIG. 11B shows plasma AST.

FIG. 12 shows plasma LDH.

FIG. 13 Increased LDH in mdx mice reported in literature from the article Murphy S, Dowling P, Zweyer M, Henry M, Meleady P, Mundegar R R, Swandulla D, Ohlendieck K. Proteomic profiling of mdx-4cv serum reveals highly elevated levels of the inflammation-induced plasma marker haptoglobin in muscular dystrophy. Int J Mol Med. 2017 June; 39(6):1357-1370. doi: 10.3892/ijmm.2017.2952.

FIG. 14 shows plasma cystatin C.

FIG. 15 shows plasma hepatoglobin.

FIG. 16 shows plasma TGF-beta.

FIG. 17 shows plasma IL-13.

FIG. 18 shows representative photomicrographics of HE-stained muscle sections.

FIG. 19 shows representative H & E images.

FIG. 20 shows decreased Inflammation Score in N-163.

FIG. 21 shows representative photomicrographics of Sirius red-stained muscle sections.

FIG. 22 shows fibrosis area (sirius red staining).

FIG. 23 shows representative photomicrographics of Masson's Trichrome-stained muscle sections.

FIG. 24 shows fibrosis area (Masson's Trichrome positive area).

FIG. 25 shows other images of Masson's Trichrome staining.

FIG. 26 shows increase in Bacteroides after N-163.

FIG. 27 shows that Faecalibacterium prausnitzii was most abundant species after N-163.

FIG. 28 shows decrease in Enterobacteriaceae after N-163 beta glucan but increase in control group.

FIG. 29 shows increase in Lactobacillus after N-163 beta glucan but decrease in control group.

FIG. 30 shows increase in Roseburia after N-163 beta glucan but decrease in control group.

FIG. 31 shows increase in Bifidobacterium after N-163 beta glucan but decrease in control group.

FIG. 32 shows increase in Prevotella after N-163 beta glucan.

FIG. 33 shows decrease in Alistipes after N-163 beta glucan but increase in control group.

FIG. 34 shows decrease in Firmicutes after N-163 beta glucan but increase in control group.

FIG. 35 shows decrease in Akkermansia muciniphila after N-163 beta glucan.

FIG. 36 shows MRC muscle power grade in percentage.

FIG. 37 shows average calcium level in serum.

FIG. 38 shows average CPK in serum.

FIG. 39 shows average ALP in serum.

. 40 FIG. 40 shows decrease in IL-6 after N-163.

FIG. 41 shows decrease in C-reactive protein (CRP) after N-163.

FIG. 42 shows increase in Bacteroides and decrease in firmicutes after N-163.

FIG. 43 shows increase in Prevotella after N-163.

FIG. 44 shows that Faecalibacterium prausnitzii abundance increased post-N-163 intervention.

FIG. 45 shows that Prevotella copri abundance increased post-N-163 intervention.

FIG. 46 shows that Bifidobacterium longum abundance increased post-N-163 intervention.

. 47 FIG. 47 shows that Streptococcus parasanguinis abundance decreased post-N-163 intervention.

FIG. 48 shows that Streptococcus salivarius abundance decreased post-N-163 intervention.

FIG. 49 shows that Parabacteroides distasonis abundance increased post-N-163 intervention.

FIG. 50 shows that Roseburia intestinalis abundance increased post-N-163 intervention.

### DETAILED DESCRIPTION OF INVENTION

The glucan used in the present invention can be a glucan derived from Aureobasidium pullulans strain APNN-M163 (Also referred to herein as “strain M163”, or “strain N-163”), and preferably β-1,3-1,6 glucan derived from N-163 (Also referred to herein simply as “N-163 glucan” or “N-163 beta glucan”). “Aureobasidium pullulans strain APNN-M163” has been deposited at the Patent Microorganisms Depositary Center, National Institute of Technology and Evaluation (Room. 122, 2-5-8, Kazusa Kamatari, Kisarazu City, Chiba, 292-0818 Japan), under the deposit number NITE P-03377, on Feb. 9, 2021.

The glucan produced by N-163 strain was estimated to have the following chemical structure (Japanese Patent Application No. 2021-187255).

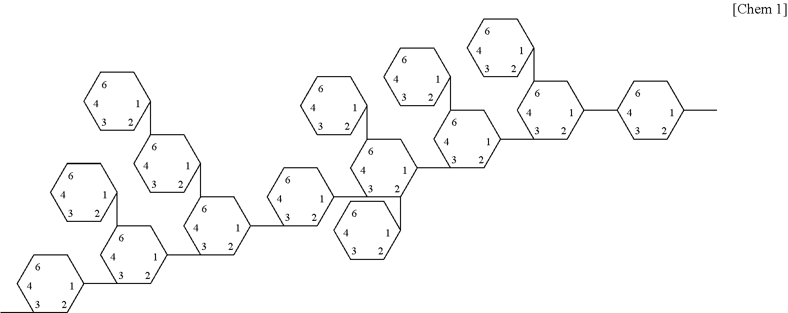

The composition of the present invention exerts its function when ingested by mammals including humans. The term “ingestion” as used herein is not limited to any administration route as long as it can enter the human body, and is realized by all known administration methods such as oral administration, tube administration, and enteral administration. Typically, oral ingestion and enteral ingestion via the digestive tract are preferable.

The dose of the present invention can be appropriately set in consideration of various factors such as administration route, age, body weight, and symptoms. The dose of the composition of the present invention is not particularly limited, but the amount of glucan is preferably 0.05 mg/kg/day or more, more preferably 0.5 mg/kg/day or more, particularly preferably 1.0 mg/kg/day. However, when ingested over a long period of time, the amount may be smaller than the preferable amount described above. In addition, the glucan used in the present invention has a sufficient dietary experience, and there is no problem in terms of safety. Therefore, an amount far exceeding the above amount (for example, 10 mg/kg/day) Or more).

The composition of the present invention can be used as a food or drink. The composition of the present invention, as a special-purpose food such as a food for specified health use and a nutritionally functional food, by administering to animals such as humans, treatment or prevention can be achieved against various diseases related to fibrosis.

When the composition of the present invention is used as food or drink, the type of food or drink is not particularly limited. Further, the shape of the food or drink is not particularly limited, and may be any shape of food or drink that is usually used. For example, it may be in any form such as solid form (including powder and granule form), paste form, liquid form and suspension form, and is not limited to these forms.

When used as a pharmaceutical, a dosage form that can be orally administered is preferable because the composition of the present invention reaches the intestine. Examples of preferable dosage forms of the drug according to the present invention include tablets, coated tablets, capsules, granules, powders, solutions, syrups, troches and the like. These various preparations are prepared according to a conventional method by using glucan, which is the active ingredient, an excipient, a binder, a disintegrating agent, a lubricant, a coloring agent, a flavoring agent, a solubilizing agent, a suspending agent, a coating agent, etc. It can be formulated by admixing the auxiliaries usually used in the technical field of pharmaceutical formulation.

In some embodiment, the present invention can be used in combination with other food, drink, drugs and any other substances in order to enhance the efficacy of the present invention.

In one embodiment, the composition or the pharmaceuticals of the present invention can improve a gut microbiota of a subject who need thereof, such as a patient of muscular dystrophy, multiple sclerosis and/or related diseases, whereby the composition or the pharmaceuticals of the present invention can improve, prevent and/or treat these diseases.

In one embodiment, improving the gut microbiota is including but not limited to a decrease in Enterobacteriaceae, an increase in Lactobacillus, an increase in Roseburia, an increase in Bifidobacterium, an increase in Prevotella, a decrease in Alistipes, a decrease in Firmicutes, a decrease in Akkermansia, or a combination thereof, and wherein the composition or the pharmaceuticals of the present invention can be used to improve, prevent and/or treat muscular dystrophy such as Duchenne muscular dystrophy (DMD).

Enterobacteriaceae, has been described as enhancing the inflammatory response and therefore its decrease will be beneficial in multiple sclerosis and DMD (Ref: https://www.mdpi.com/2076-2607/9/4/697/htm).

Restoration of Lactobacillus species has been shown to decrease inflammatory cytokines and its increase by N-163 is beneficial in MS (Ref: https://www.mdpi.com/2076-2607/9/4/697/htm).

Increase in Roseburia is a marker of intestinal health as it is a butyrate (a beneficial metabolite) producing bacteria (Ref: Future Microbiol 2017: 157-170).

Bifidobacterium longum has been reported to increase both muscle function and cognitive ability (Ref: https://www.ncbi.nlm.nih.gov/pmc/articles/PMC7745561/). The increase in Bifidobacterium longum after N-163 is therefore beneficial.

Alistipes contributes to inflammation and epithelium alterations (https://www.ncbi.nlm.nih.gov/pmc/articles/PMC7745561/). Firmicutes, plays a role in the onset of depression via affecting the inflammation levels of host (https://www.frontiersin.org/articles/10.3389/fcimb.2022.831186/). Increase in the abundance of Akkermansia has been reported in patients with PD and MS (https://pubmed.ncbi.nlm.nih.gov/28843021/) Therefore the decrease of these bacteria is beneficial.

In another embodiment, improving the gut microbiota is including but not limited to an increase in Bacteridetes, a decrease in Firmicutes, an increase in Prevotella, an increased in Faecalibacterium prausnitzii, an increase in Prevotella copri, an increase in Bifidobacterium longum, a decrease in Streptococcus parasanguinis, a decrease in Streptococcus salivarius, an increase in Parabacteroides distasonis, an increase in Roseburia intestinalis, or a combination thereof, and wherein the composition or the pharmaceuticals of the present invention can be used to improve, prevent and/or treat multiple sclerosis.

MS patients have presented gut dysbiosis with a reduction in bacteria belonging to the Prevotella genus especially Prevotella copri (https://pubmed.ncbi.nlm.nih.gov/30513004/; https://www.ncbi.nlm.nih.gov/pmc/articles/PMC5730390/). Fecalibacterium which is an anti-inflammatory commensal bacterium has been reported to be low in patients with MS (J Investig Med. 2015 June; 63(5):729-34. doi: 10.1097/JIM.0000000000000192.). Bifidobacteria has substantial roles in regulation of immune response and lower frequency of bifidobacteria in gut of MS patients has been reported (https://www.sciencedirect.com/science/article/abs/pii/S2211034819303554) Parabacteroides distasonis to be reduced in abundance in the MS patients (https://www.pnas.org/doi/10.1073/pnas.1711235114). R. intestinalis has been shown to prevent intestinal inflammation (https://www.frontiersin.org/articles/10.3389/fcimb.2021.757718/full). Therefore, the increase of these bacteria after N-163 is beneficial.

Streptococcus parasanguinis (www.pnas.org/cgi/doi/10.1073/pnas.2011703117), S. salivarius/thermophilus (www.pnas.org/cgi/doi/10.1073/pnas.2011703117) has been reported to be significantly increased in MS patients. Therefore, the decrease of these bacteria after N-163 is beneficial.

### EXAMPLES

### Example 1

### Methods

This trial was an investigator-initiated, single-centre, randomized, open-label, prospective, comparative, two-arm clinical study of patients with DMD. The study was conducted over 45 days. The two treatment arms included

Treatment arm I, control arm: Conventional treatment regimen comprising standard routine physiotherapy for joint mobility along with medications, viz., T. calcium and vit. D 1000 with or without T. deflocort (steroid) 6 mg to 24 mg.

Treatment arm II, intervention: One sachet of N-163 beta glucan (15 g gel) once daily along with conventional treatment.

Inclusion criteria: Male subjects with molecular diagnosis of DMD aged 6-18 years who were willing to participate in the study with written informed consent.

Exclusion criteria: Patients with a previous (within the past 1 month) or concomitant participation in any other therapeutic trial; a known or suspected malignancy; any other chronic disease or clinically relevant limitation of renal, liver or heart function according to the discretion of the investigator.

### Investigations:

The following tests were carried out after written consent was obtained from the study subjects.

At baseline and at the end of the study (after 45 days):
Background survey: gender, date of birth, age, habits, current medical history, medication, treatment, allergies (to drugs and food), regular use of food for specified health uses, functional foods, health foods, intake of foods rich in β-glucan foods containing beta-glucan and intake of immunity-boosting foods
Medical history and physical measurements: height, weight, BMI, temperature
Physiological examination: systolic blood pressure, diastolic blood pressure, pulse rate
ECG
Muscle strength test using MRC grading [10]
Six-minute walk test (6MWT) [11]
North Star Ambulatory Assessment (NSAA) [12]
Blood sampling and investigations for the levels of IL-6, IL-13, TGF-β, creatinine kinase (CK), titin, haptoglobin, TNF-α, dystrophin, cystatin in the blood and myoglobin in the urine
Subjects were contacted every week for drug compliance and recording of adverse effects, if any
Study subjects=28

The study was designed as an exploratory study, so there were two intervention conditions: one control and one test group. As the minimum number of participants required for statistical comparisons within and between intervention conditions is four per intervention condition, a total of 28 target study participants (10 in treatment arm I [control] group and 18 in treatment arm II [N-163]) were used.

### Selection of Study Subjects

Study investigators and other investigators included study subjects who had consented to participate in the study, met the selection criteria and not the exclusion criteria, and who were judged to have no problem participating in the study.

### Allocation of Study Subjects

The person in charge of the allocation, as specified in the study protocol, allocated the study subjects to the two groups by simple randomization.

### Primary Outcome:

Observation of changes in the levels of IL-6 and myoglobin urea from the baseline.

### Secondary Outcome:

Observation of changes in the levels of IL-6, IL-13, TGF-β, CK, titin, dystrophin, haptoglobin, cystatin C, and TNF-α in the serum and urine myoglobin levels measured by ELISA.
Monitoring for adverse effects

### Statistical Analysis:

All data were analysed using Excel statistics package analysis software (Microsoft Office Excel®); Student's t-tests and ANOVA were used. When there was a significant main effect, post hoc pairwise comparisons were performed, and p-values <0.05 were considered significant.

### Results

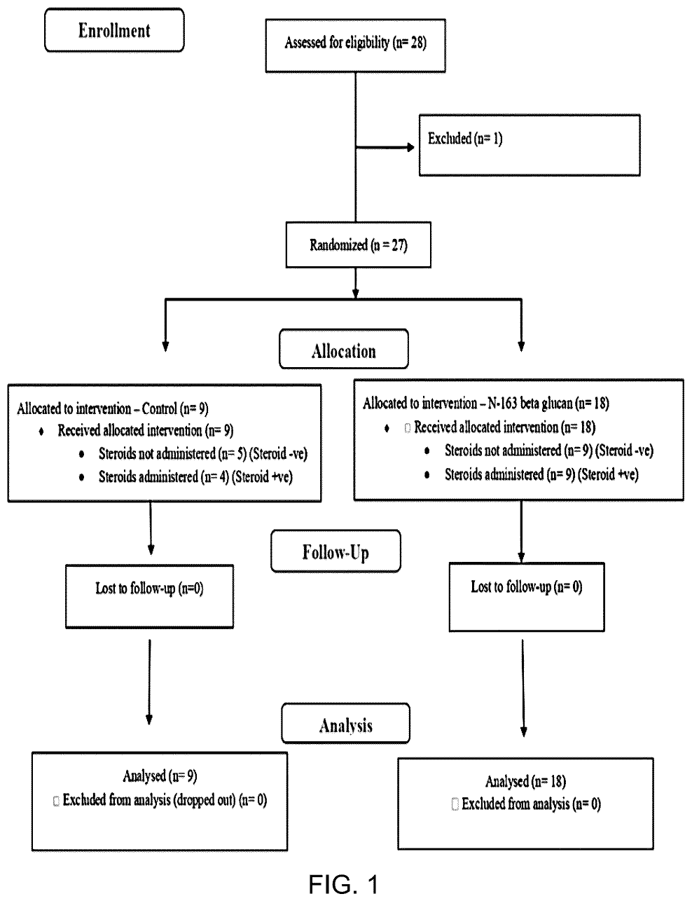

Twenty-eight patients were screened and 27 were randomized to control (n=9) and treatment (n=18). One patient was disqualified due to misrepresentation of diagnosis. The CONSORT flow diagram of the trial is shown in FIG. 1.

Demographics are shown in Table 1. The mean±SD age for the total study population was 11.18±3.86 years (range 5-19 years) and was similar across the groups. The mean±SD body weight was 35.59±15.5 kgs (range=10 to 65 kgs).

TABLE 1
Table 1: Demographics and baseline characteristics

| | | Age in | Weight | Exon | Ambulatory/ |
|---|---|---|---|---|---|
| | Subject | years | in kgs | Deletion | Non-Ambulatory |
| Control | 1 | 7 | 12 | 10 & 11 | Ambulatory |
| Steroid −ve | 2 | 10 | 27 | 46-55 | Non-Ambulatory |
| | 3 | 19 | 48 | | Ambulatory |
| | 4 | 14 | 60 | 8 to 48 | Non-Ambulatory |
| | 5 | 15 | 55 | 10-11/Dup | Non-Ambulatory |
| Control | 1 | 19 | 65 | 44 | Non-Ambulatory |
| Steroid +ve | 2 | 7 | 25 | 5 & 6 | Ambulatory |
| | 3 | 7 | 21 | 49-52 | Ambulatory |
| | 4 | 13 | 48 | 52 | Ambulatory |
| N-163 | 1 | 10 | 28 | 48-50 | Ambulatory |
| Steroid −ve | 2 | 4 | 10 | 45-50 | Ambulatory |
| | 3 | 13 | 38 | 45-50 | Ambulatory |
| | 4 | 10 | 45 | 60 | Ambulatory |
| | 5 | 7 | 39 | 10-11/Dup | Non-Ambulatory |
| | 6 | 8 | 21 | 49-52 | Ambulatory |
| | 7 | 13 | 39 | 43 | Non-Ambulatory |
| | 8 | 14 | 40 | | Non-Ambulatory |
| | 9 | 7 | 22 | 44-57/Dup | Ambulatory |
| N-163 | 1 | 5 | 18 | 48-50 | Non-Ambulatory |
| Steroid +ve | 2 | 14 | 59 | 17 | Ambulatory |
| | 3 | 15 | 40 | 60 | Ambulatory |
| | 4 | 10 | 20 | 48-52 | Ambulatory |
| | 5 | 10 | 36 | 48-50 | Ambulatory |
| | 6 | 14 | 58 | 18-29 | Non-Ambulatory |
| | 7 | 10 | 22 | 48-52 | Ambulatory |
| | 8 | 12 | 36 | 48-52 | Non-Ambulatory |
| | 9 | 15 | 29 | 07-Jun | Ambulatory |

The distribution of patients was as follows:
Group I: Control group (n=9);
A. Steroids not administered (n=5) (Steroid −ve)
B. Steroids administered (n=4) (Steroid +ve)
Group II: Treatment (N-163) group (n=18);
A. Steroids not administered (n=9) (Steroid −ve)
B. Steroids administered (n=9) (Steroid +ve)

No adverse events were reported. No clinically significant changes from baseline data were observed on physical examination or in vital signs-temperature, blood pressure, oxygen saturation, pulse rate or ECG (data not shown).

### Biomarker Levels:

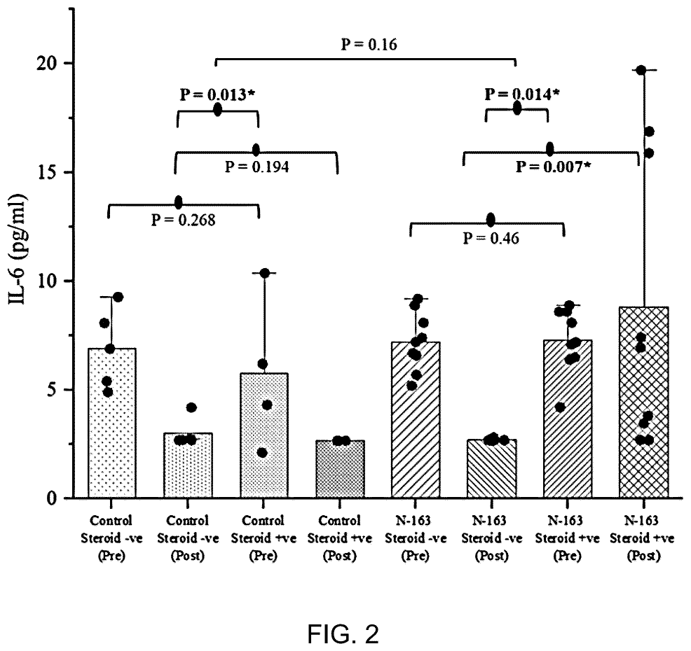

Levels are expressed as mean±SD. IL-6 showed the highest decrease in the N-163 Steroid −ve group, from a baseline value of 7.2±1.2 pg/ml to 2.7±0.03 ng/ml post intervention, but the difference was not significant (p-value=0.16) (FIG. 2).

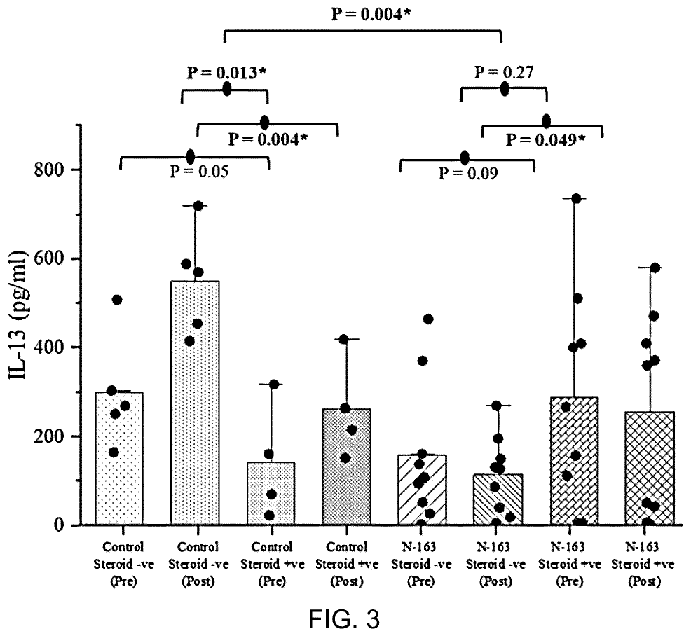

IL-13 increased in both control groups—from 300.4±114.5 pg/ml at baseline to 550.732±107.95 pg/ml post-intervention in the Steroid −ve group and from 142±112.82 pg/ml at baseline to 263.5±99.38 pg/ml post-intervention in the Steroid +ve group. It decreased in both the treatment groups—from 157.76±148.68 pg/ml at baseline to 114.08±81.5 pg/ml post-intervention and from 289.56±232.88 pg/ml at baseline to 255.56±214.13 pg/ml post-intervention. The difference was statistically significant (p-value=0.004) (FIG. 3).

FIG. 3: IL-13 levels showed a statistically significant increase in control groups but decreased in treatment groups (*p-value significance <0.05)

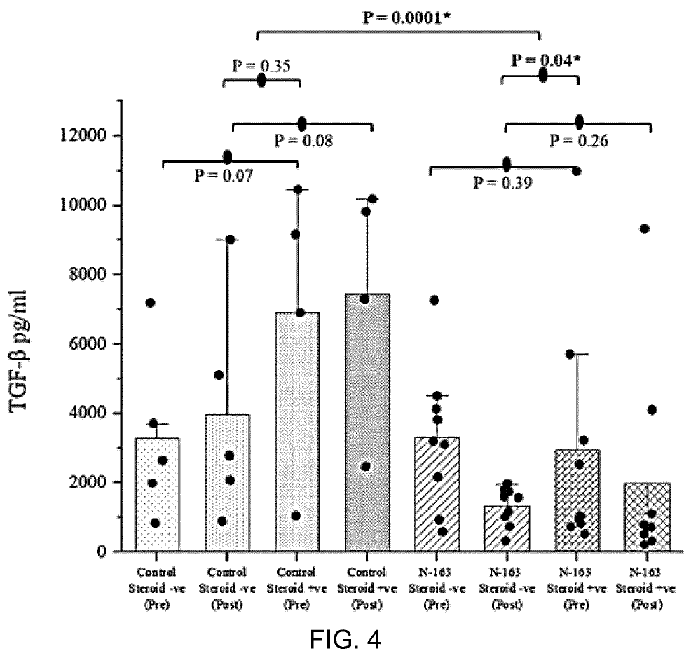

TGF-β levels showed a significant decrease in the N-163 Steroid −ve group, from a baseline value of 3302±1895 ng/ml to 1325.66±517 ng/ml post intervention, which was significantly lower than all the other groups (p-value=0.0001) (FIG. 4).

Dystrophin levels showed a significant increase in the N-163 Steroid −ve group, from a baseline value of 3.01±1.58 ng/ml to 4.01±1.44 ng/ml post intervention, and the N-163 Steroid +ve group went from a baseline value of 3.15±2.43 ng/ml to 3.78±2.17 ng/ml post intervention, which was significantly higher than the control groups (p-value=0.0009) (FIG. 2). The N-163 Steroid −ve group showed higher dystrophin expression than the N-163 Steroid +ve group, but the difference was not significant (p-value=0.11). The percentage increase in dystrophin levels in the treatment group was up to 32.8%.

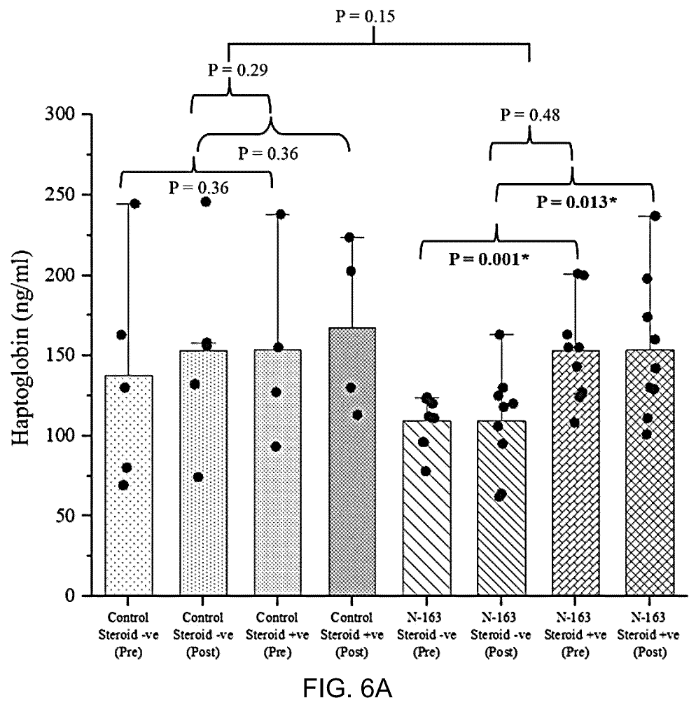

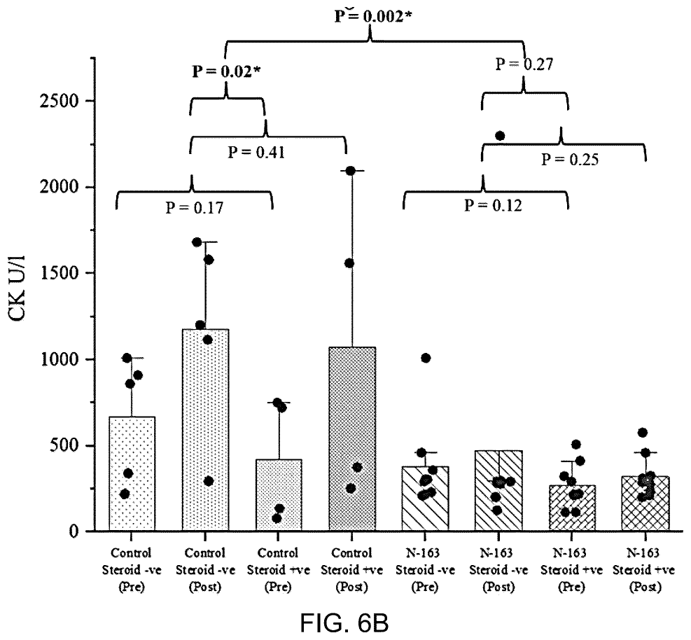

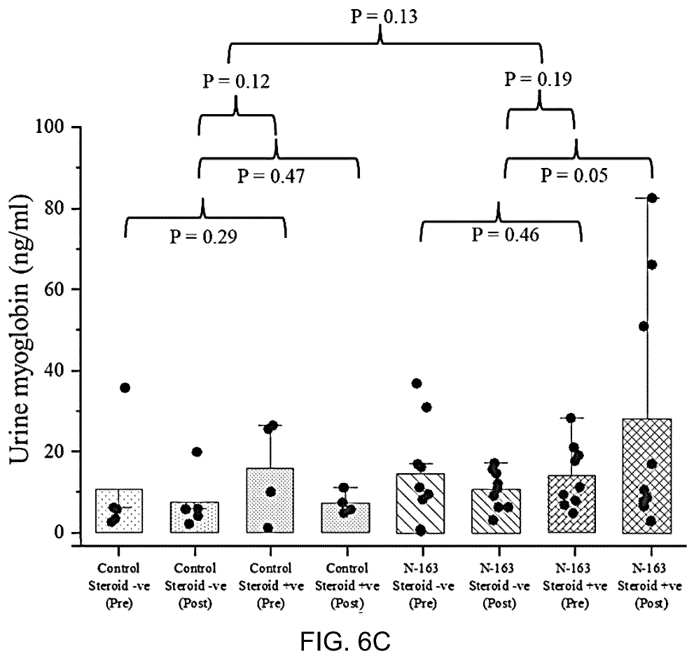

Haptoglobin did not show much difference pre or post intervention in the treatment groups, but it was marginally increased in the control group (FIG. 6A). CK increased in the treatment groups (FIG. 6B). Urine myoglobin increased in the N-163 Steroid +ve group but decreased in all the other groups (FIG. 6C).

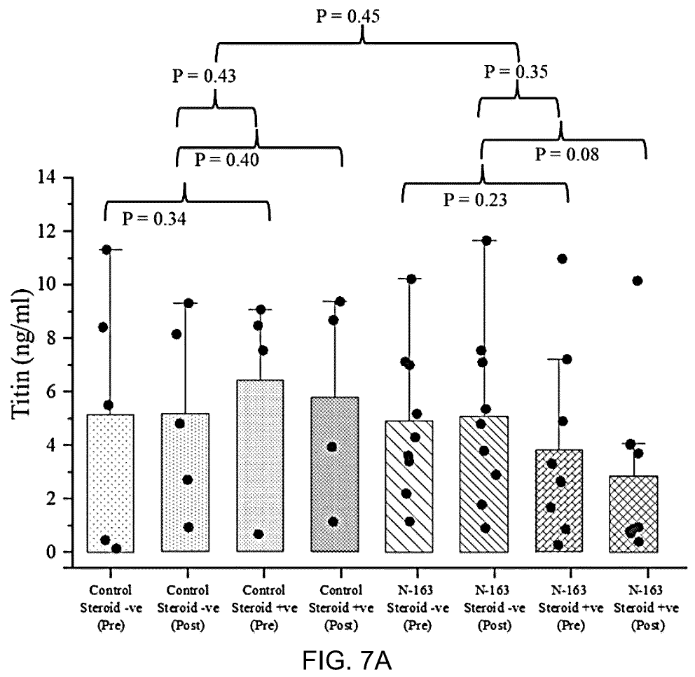

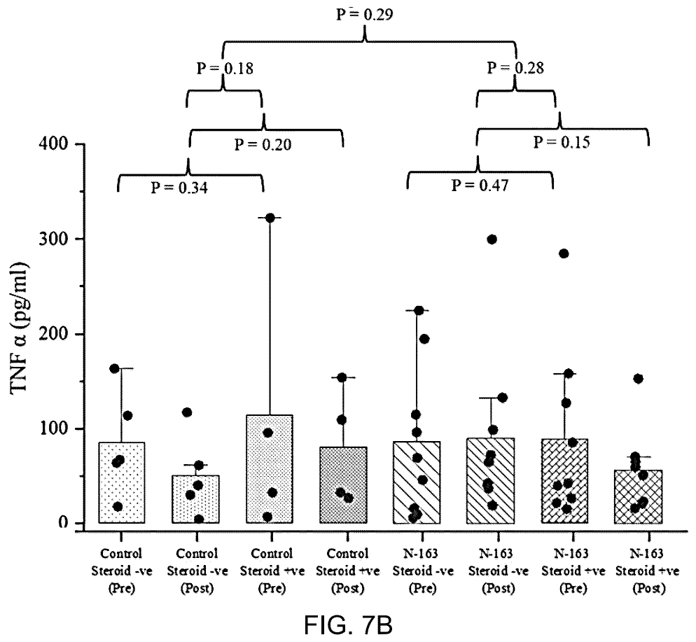

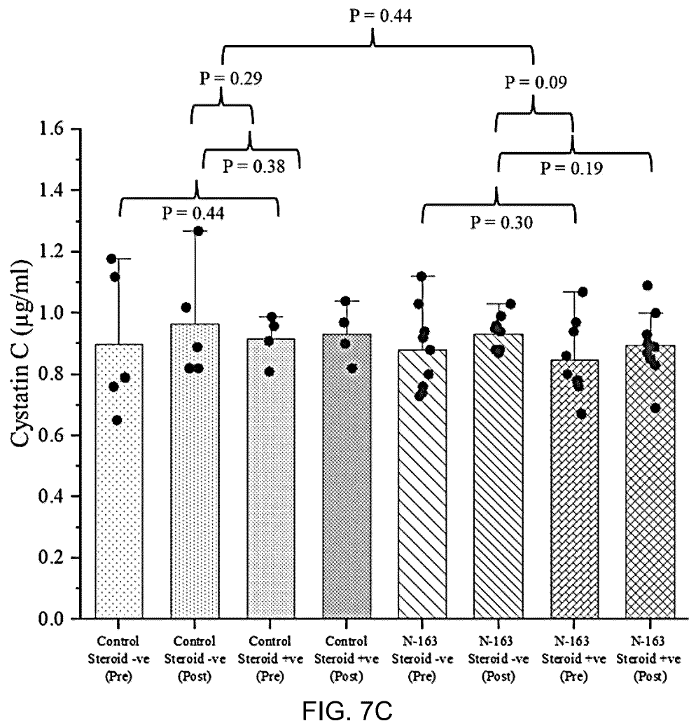

Titin and cystatin C decreased in the N-163 Steroid +ve group and the control Steroid +ve group, but the difference was not significant (FIG. 7A.B). TNF-α decreased in all the groups except the N-163 Steroid −ve group.

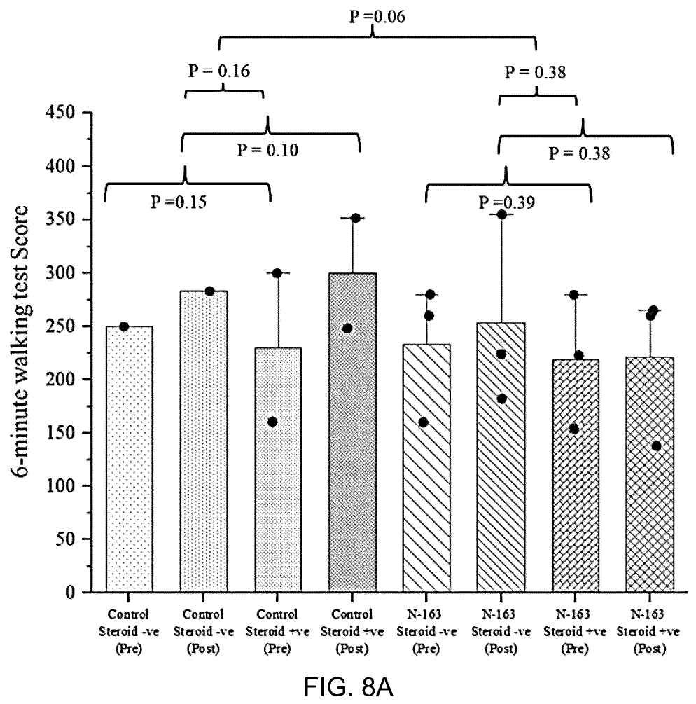

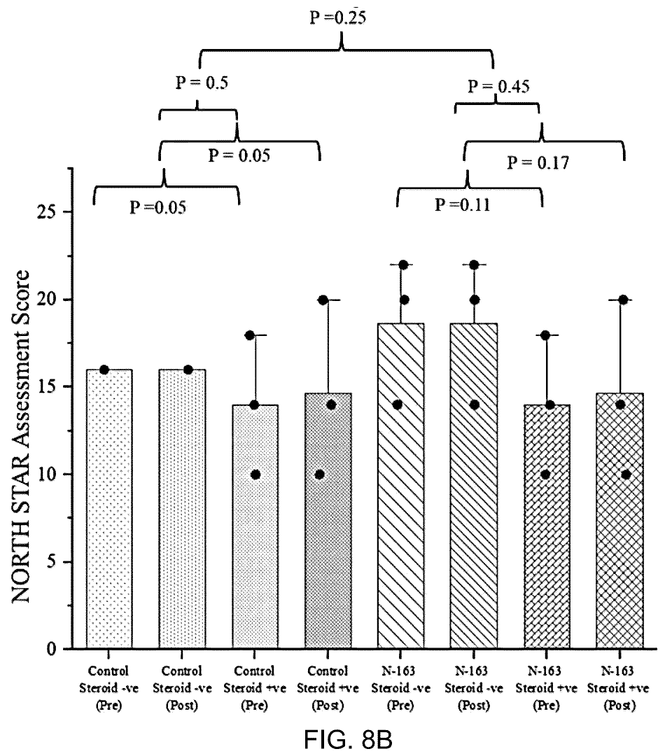

The 6MWT and NSAA did not show any significant differences between the groups (FIG. 8 A, B). The MRC grading showed improvement in 12 out of 18 patients (67%) in the treatment group and only four out of nine (44%) subjects in the control group (Table 2).

TABLE 2
Table 2: Medical research council (MRC) grading
of muscle power in the study groups

| | | | | |
|---|---|---|---|---|
| | Ambulatory/ | | Post- | |
| S. No | Non-ambulatory | Baseline | intervention | Progression |

Control group

| | | | | |
|---|---|---|---|---|
| 1 | Ambulatory | 151 | 165 | Improved |
| 2 | Ambulatory | 133 | 162 | Improved |
| 3 | Non-ambulatory | 70 | 80 | Improved |
| 4 | Non-ambulatory | 164 | 164 | No change |
| 5 | Non-ambulatory | 109 | 106 | Worsened |
| 6 | Non-ambulatory | 102 | 105 | Improved |
| 7 | Non-ambulatory | 117 | 115 | Worsened |
| 8 | Non-ambulatory | 110 | 109 | NO |
| 9 | Non-ambulatory | 105 | 102 | Worsened |

Treatment Group

| | | | | |
|---|---|---|---|---|
| 1 | Ambulatory | 134 | 133 | Worsened |
| 2 | Ambulatory | 146 | 163 | Improved |
| 3 | Ambulatory | 119 | 131 | Improved |
| 4 | Ambulatory | 127 | 136 | Improved |
| 5 | Ambulatory | 154 | 160 | Improved |
| 6 | Ambulatory | 158 | 168 | Improved |
| 7 | Non-ambulatory | 102 | 119 | Improved |
| 8 | Non-ambulatory | 116 | 117 | Improved |
| 9 | Non-ambulatory | 127 | 127 | No change |
| 10 | Non-ambulatory | 120 | 131 | Improved |
| 11 | Non-ambulatory | 96 | 107 | Improved |
| 12 | Non-ambulatory | 111 | 122 | Improved |
| 13 | Non-ambulatory | 117 | 113 | Worsened |
| 14 | Non-ambulatory | 108 | 108 | No change |
| 15 | Non-ambulatory | 103 | 107 | Improved |
| 16 | Non-ambulatory | 145 | 148 | Improved |
| 17 | Non-ambulatory | 93 | 119 | Improved |

### Discussion

Current interventions for DMD, such as corticosteroids and rehabilitative care, help to prolong survival up to the third or the fourth decade of life. Corticosteroids remain the mainstream supportive approach to slow inflammation and the associated decline in muscle strength and function [4]. However, steroids have their own adverse effects, and their prescription is based on risk versus benefit to that specific patient and tolerance to the medication. Exon-skipping gene therapy and cell-based strategies to replace the mutant DMD gene are in development, but the desired outcome has not yet been achieved. In the meantime, nutraceuticals can be considered potential strategies for immune modulation and alleviating inflammation, as they are safer with lesser adverse effects [4]. Improvement of the locomotor performances and mitochondrial respiration by 1,3-1,6 beta-glucans in zebra fish model of muscular dystrophy [6] has already been reported.

In the current study, we focussed on a 1-3,1-6 beta glucan from the N-163 strain of the black yeast A. pullulans that has been reported to mitigate inflammation, evidenced by a decrease in anti-inflammatory markers and production of beneficial immuno-modulation [6-8]. The safety profile of N-163 beta glucan has been confirmed by the results.

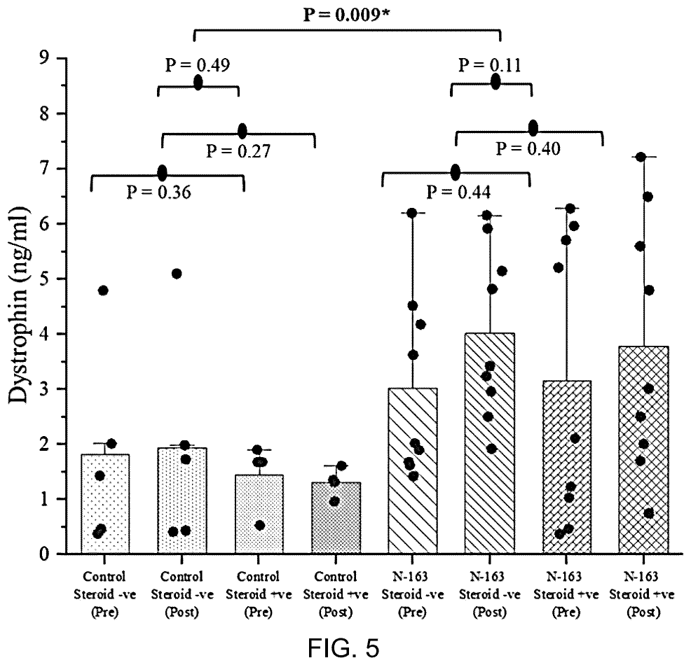

Anti-inflammatory and anti-fibrotic outcomes: Circulating IL-6 is chronically elevated in individuals with DMD [13], which has been reported to contribute to DMD-associated cognitive dysfunction. IL-6 blockades have been advocated as a therapeutic approach for DMD [14]. In the present study, IL-6 showed highest decrease in the N-163 Steroid −ve group (FIG. 2). While IL-6 is an acute inflammatory biomarker (14), IL-13 is a pro-fibrotic biomarker [15] and was significantly decreased (FIG. 3). Together with the TGF-β pathway, it is a major proinflammatory and pro-fibrotic cytokine responsible for the chronic inflammatory response leading to replacement of the muscle by scar tissue or fibrosis, resulting in muscle weakness and loss of muscle function [16]. TGF-β levels also showed a significant decrease in the N-163 Steroid −ve group (FIG. 4). Dystrophin restoration of 20% expression [17,18] is considered the point of efficacy for a DMD therapy [19] and was found to increase by 32.8% in both the treatment groups (FIG. 5) of the present study from baseline. This establishes N-163 beta glucan as an efficient agent for DMD. This dystrophin increase could be attributed to the immune modulation proven through control of anti-inflammatory and anti-fibrotic markers (IL-6, IL-13 and TGF-3).

Other biochemical markers of relevance: While haptoglobin and urine myoglobin did not show significant differences, the increase in urine myoglobin in the N-163 Steroid +ve group deserves more analysis concerning the underlying mechanism. Greater activity among steroid-treated individuals may place their dystrophin-deficient muscles under greater mechanical stress, predisposing them to further muscle fibre damage and consequent myoglobinuria [20]. While titin and cystatin C decreased in the N-163 steroid +ve group and in the control Steroid +ve group, there was an increase in CK, which is paradoxical, as reports suggest that titin concentration correlates significantly with serum CK concentration [21].

Muscle strength evaluation: There were three evaluations to assess muscle strength and tone, done in a blinded manner by the same physiotherapist at baseline and post intervention. Though the 6MWT and NSAA did not show any significant differences between the groups, MRC grading showed improvement of muscle strength in 67% of the subjects in the treatment group compared to 44% subjects in the control group, which is significant. The limitation of this being a 45-day study is relevant to the muscle-strength and functional evaluations, mandating the need for a longer study and follow-up duration. However, though small, the improvement in MRC grading at 45 days could be again attributed to the immune modulation effects of this disease-modifying supplement. The study shows proof of concept that DMD could be tackled by the N-163 beta glucan from three aspects: decrease in inflammation shown by decreased IL-6 and TNF-α, decrease in fibrosis evident by decreased TGF-β and IL-13 and, more importantly, restoration of dystrophin evident from a 32.8% increase in dystrophin levels. These effects hold regardless of the use or non-use of steroids, which is important, as this safety-proven food supplement can help DMD patients regardless of steroid status.

Chronic inflammation being common to pathogenesis of all muscular dystrophies, immunomodulatory treatment may benefit patients with diverse types of muscular dystrophy [22]. Further, modulating the inflammatory response and inducing immunological tolerance to de novo dystrophin expression is critical to the success of dystrophin-replacement therapies [23]. The need to evaluate the muscles involved in respiratory function and myocardium should be mentioned here, as they are the cause of mortality in most of the DMD patients [1]. Though other dystrophinopathies, such as limb girdle muscular dystrophy (LGMD), do not involve respiratory or cardiac muscles, inflammatory overactivity is the common pathophysiology among types of muscular dystrophy [22]. Once proven efficacious for DMD, extending the beneficial application of the N-163 beta glucans to other dystrophinopathies such as LGMD can be considered.

DMD is a rare genetic disease with a maximum life expectancy of up to fourth decade, with the majority of victims dying in their late twenties to thirties. The average lifespan at birth, which was 20+ years for those born in or before 1970, has gradually increased by 10-15 years for those born and diagnosed with DMD in the 1980s and 1990s. This increase is attributed to better or early ventilatory assistance, steroid usage and cardiac care [24,25], which are only supportive interventions. With the gene therapies approved recently, there is a hope of additional progress and increase in lifespan [26]. Though these gene therapies (such as exon skipping) address the root cause by splicing out selected exons from the pre-mRNA at or next to the mutation site, generating a translatable transcript from the mutant dystrophin gene leading to dystrophin expression [26, 27], they are still marred by challenges such as delivery of gene-editing components throughout the musculature and mitigation of possible immune responses [28]. The current need, therefore, is to modulate the immune system and control the inflammation and ensuing fibrosis to delay the progression of the disease. The earlier usage of steroids in a regular manner was later changed to intermittent usage [29] with regimens varying between institutes; now, newer steroids with lesser adverse effects are in various stages of progress towards clinical applications [30]. In this background, the safety of this N-163-produced beta glucan food supplement without adverse reactions is to be considered an indispensable value addition. Targeting the inflammation component (the criteria for selecting this supplement for this study) having yielded beneficial outcomes, additional studies on this characteristic could be of value to possibly extending their application for other neuroinflammatory diseases, such as multiple sclerosis. At this point, it is essential to mention the gut microbiome for two reasons; one being the association of the microbiome with the severity of neuroinflammatory conditions such as multiple sclerosis [31], and another being the fact that beta glucans have been reported to yield beneficial reconstitution of the gut microbiome in earlier studies [32] in children with autism spectrum disorder, a neurodevelopmental disease. For both multiple sclerosis and DMD, steroids to suppress inflammation are common, but associated implications for gut microbiota in DMD have not been reported often and are worthy of future study.

The limitations of the study include uneven distribution of subjects and short follow-up (only 45 days); improvements in muscle function over the course of the study showed variability that may have been due to the level of sensitivity to change of functional assessments during the disease progression in the age group. Among the 27 subjects, two-thirds were ambulatory and the remaining non-ambulatory; the evaluation criteria differences must be kept in mind, which may show equivalent quantification among all DMD patients at different stages of disease severity when non-invasive myograms to measure the individual muscles accurately could be undertaken. Further, consumption of steroids vs those who did not consume them or those who had stopped steroids after an initial duration of consumption, as well as regimen variation, are to be considered while interpreting the outcomes. All these aspects mandate the need for larger randomized clinical trials of longer duration to validate this supplement as a treatment.

### Example 2

Re-examining the therapeutic management of muscular dystrophies from a vascular smooth muscle dystrophin-centred approach:

Duchenne muscular dystrophy (DMD) has long been believed to be the result of skeletal and cardiac muscle wasting due to the absence of dystrophin in the sarcolemma of these muscle fibres. This absence of dystrophin is caused by mutations in the dystrophin gene [33,34]. Most of the treatment approaches for DMD have primarily addressed the pathophysiology of skeletal muscles, even though there are well-established vascular smooth muscle defects associated with DMD [33,34]. The pathophysiology of DMD is considered to develop due to the loss of dystrophin in skeletal and cardiac muscles which destabilizes a highly organized complex of transmembrane and cytosolic proteins that forms a structural link between the extracellular matrix and the intracellular actin cytoskeleton. Destabilization of this complex leads to increased susceptibility to contraction-induced damage in muscle cells, inflammation, failed muscle regeneration, and the progressive replacement of muscle by fibrotic tissue and fat [35]. The currently available gene replacement and exon-skipping therapies have not yielded the expected outcome. Previous research attributes this to a lack of understanding of the precise mechanism by which skeletal muscle dystrophin deficiency produces the clinical phenotype [33,34]. Recently, strong emerging evidence [36] has indicated that the primary cause of DMD is a lack of dystrophin in the smooth muscle of blood vessels and not skeletal or cardiac muscle. This dystrophin deficiency in vascular smooth muscle affects nitric oxide (NO) production and vasodilation. Thus, it restricts the blood supply and thereby results in muscle ischaemia, injury and fatigue during exercise, which can lead to fibrosis in the long term [34]. This revelation is important for all aspects of DMD, from diagnosis to management. The clinical diagnosis of DMD is based on symptoms, markers in the blood, such as elevated creatine kinase, genetic analysis, and biopsy of skeletal muscles for dystrophin assessment [35]. Since the primary pathology has been indicated to be vascular smooth muscle dystrophin deficiency, a diagnosis based only on the evaluation of the skeletal muscle may not suffice. Furthermore, a homologue of dystrophin that is expressed on the blood vessel membrane and is not encoded by the dystrophin gene has also been reported [37].

The disease progression of DMD is directly proportional to the activity of the musculature. Vascular smooth muscles are in constant movement with pulsatile blood flow, which is not the case for skeletal muscles. This aspect indicates that vascular smooth muscles could be a primary contributing factor to the faster progression of the disease. In contrast to skeletal muscle, vascular muscle can switch to a synthetic, largely noncontractile phenotype in response to proinflammatory stimuli, diet or other factors [38], such as the development of atherosclerosis, which will also affect the disease phenotype in DMD. Thus, it can lead to faster disease progression as age advances. There are sex differences in DMD, with females being carriers and males being primarily affected by the disease. These differences can also be attributed to the anti-atherosclerotic effects of oestrogen and differences in nitric oxide (NO) synthesis from the vascular endothelium in premenopausal women [39]. The involvement of vascular smooth muscle dystrophin in the dystrophin-glycoprotein complex (DGC) and its association with the anchorage proteins of the complex, such as NO-synthase (nNOS), aquaporin-4 (AQP4) and acetylcholine receptors, make DMD a systemic disease that requires systemic intervention rather than skeletal muscle disease-targeted intervention [39]. The “functional ischaemia” perspective of this disease, rather than the muscular fatigue that contributes to the disease phenotype, indicates the importance of addressing ischaemia for an effective therapy, both existing and futuristic, to achieve the expected outcome [39]. In addition to the above alterations in the smooth muscle cells and endothelium of blood vessels, muscle stem cells, also known as satellite cells, from DMD mice have also been shown to have a reduced capacity to promote angiogenesis. These satellite cells promote angiogenesis under normal conditions, as they promote the secretion of vascular endothelial growth factor (VEGF) and hypoxia-inducible factor-1α (HIF-1α), which promote angiogenesis. In dystrophic satellite cells, the levels of VEGF and hypoxia-inducible factor-1α (HIF-1α) were found to be significantly decreased [40]. Therefore, restoration of VEGF expression has been undertaken as one of the key angiogenesis-promoting strategies in DMD. Virus-based gene therapies, such as VEGF overexpression, the direct intramuscular delivery of growth factors, the inhibition of vascular growth factor receptor 1 (VEGFR-1), which is a negative regulator of angiogenesis, modulation of the NO signalling pathway either via I-arginine and NO donor supplementation, genetic nNOS overexpression and 5-phosphodiesterase (PDE5) are some therapies being researched for angiogenesis promotion aimed at managing DMD through a vascular smooth muscle-dystrophin centred approach [40].

Other systemic approaches employing PPAR agonists are also being researched for DMD therapeutics which are predominantly smooth muscle centred anti-fibrotic agents. The rationale behind this is that lipid dysregulations that cause atherosclerosis contribute changes in smooth muscle dystrophin. Additionally, peroxisome proliferator-activated receptors (PPARs), which regulate genes that are involved in development, metabolism, inflammation, and many cellular processes, are also involved in the deposition of fat in these muscles [41]. Activation of PPARβ/δ and its regulation of Utrophin A, which compensates for dystrophin deficiency, have been reported to ameliorate the DMD phenotype in X-linked muscular dystrophy (mdx) mice [41].

Another factor to investigate in the systemic implications of smooth muscle dystrophin deficiency is the contribution of the autonomous nervous system to fibrosis from functional ischaemia [38]. Additionally, gastrointestinal motility disorders, such as delayed gastric emptying, decreased intestinal transit and chronic constipation, in combination with a lack of dystrophin can cause an increase in NF-κB expression. This can cause a decrease in the contractile phenotype and smooth muscle functions in the intestine, which have been reported in DMD patients [41]. All these findings indicate another important target for therapeutic strategies on a systemic scale for DMD: the gut microbiome-brain axis. Recent studies have highlighted the importance of the interactions of gut microbiota and PPARs in skeletal muscle pathologies. Treatment of mice with the antibiotic drug metronidazole led to an increase in proteobacteria, resulting in skeletal muscle atrophy. In these mice, changes associated with the circadian clock machinery in peripheral muscles and PPARγ [41] were observed, suggesting a possible link between gut dysbiosis and the muscle chrono-metabolism phenotype.

Therefore, a therapeutic strategy that can address several of these smooth muscle and vascular dysfunctions along with gut dysbiosis could serve as an effective agent or adjunct to existing therapies. Our recent clinical trial [42] on biological response modifier glucans (BRMGs) in young boys with DMD indicated an increase in the plasma levels of dystrophin, which is directly derived from vascular smooth muscle. This is a step forward in addressing the vascular smooth muscle component of pathophysiology in muscular dystrophy. This study also reported a decrease in IL-6 and TGF-β and an improvement in muscle strength and a six-minute walking test. These findings could be attributed to the multipronged potentials of these BRMGs in beneficially regulating lipid metabolism, PPAR agonist action and immune-modulation. Further research is warranted to evaluate the potential of such systemically acting agents from a vascular smooth muscle-centred approach.

### Conclusion

N-163 beta glucan with and without steroids helped decrease IL-6, TGF-β and IL-13 and increase dystrophin levels along with improvement of muscle strength in subjects with DMD in this clinical study. Thus, N-163 beta glucan is a safe and effective potential therapeutic disease-modifying adjunct for patients with DMD. While the benefits documented may help slow the rate of progression of this devastating disease, confirmation by longer and larger studies will help establish this agent for routine clinical application as a disease-modifying agent with the potential to help prolong the lifespan of DMD patients. After such validation, extending its application to other dystrophinopathies such as LGMD could be considered, and further in-depth research on gut microbiomes and their implications in neuroinflammatory diseases are likely to shed light on the mechanism of action, leading to additional beneficial applications.

Background: Duchenne muscular dystrophy (DMD) is an inherited neuromuscular disorder causing progressive muscle weakness and premature death. Steroids remain the mainstream approach for supportive care but have side effects; other targeted therapies and gene therapies are also being developed. As there is limited evidence on the use of disease-modifying nutritional supplement adjuncts in DMD, this pilot trial is to evaluate the effects of supplementation of Aureobasidium pullulans-derived 1,3-1,-6 beta glucan from the N-163 strain in young patients with DMD.

### Methods

Twenty-seven patients with Duchenne muscular dystrophy (DMD)—nine in the control arm (undergoing conventional therapies)—participated. The patients were divided into groups: those not administered steroids (Steroid −ve) (n=5), those administered steroids (Steroid +ve) (n=4), and 18 in the treatment arm (N-163 beta glucan supplement along with conventional therapies; N-163 Steroid −ve and N-163 Steroid +ve); they participated in the study for 45 days. Assessments of muscle function, disease status, and levels of IL-6, IL-13, TGF-β, creatinine kinase (CK), titin, TNF-α, haptoglobin, and dystrophin in the blood and myoglobin in the urine were performed at baseline and at the end of the study.

### Results

IL-6 showed a significant decrease in the N-163 Steroid −ve group, from a baseline value of 7.2±1.2 pg/ml to 2.7±0.03 ng/ml. IL-13 decreased in both treatment groups—from 157.76±148.68 pg/ml to 114.08±81.5 pg/ml (N-163 Steroid −ve) and from 289.56±232.88 pg/e to 255.56±214.13 pg/ml (N-163 Steroid +ve). TGF-β levels showed a significant decrease in the N-163 Steroid −ve group, from a baseline value of 3302±1895 ng/ml to 1325.66±517 ng/ml post intervention. Dystrophin levels increased by up to 32% in both Steroid +ve and −ve groups. Medical research council (MRC) grading showed muscle strength improvement in 12 out of 18 patients (67%) in the treatment group and four out of nine (44%) subjects in the control group.

### Conclusion

Supplementation with the N-163 beta glucan food supplement produced disease-modifying beneficial effects: a significant decrease in inflammation and fibrosis markers, increase in dystrophin and improvement in muscle strength in DMD subjects over 45 days, thus making this a potential adjunct treatment for DMD after validation. A longer duration of follow-up and further research on the mechanism of action and commonalities with other diseases provoked by hyperactive inflammation and/or fibrosis may pave the way for their extended applications in other dystrophinopathies and neuroinflammatory diseases.

Trial registration: Clinical trials registry of India, CTRI/2021/05/033346. Registered on 5 May 2021.

### Example 3: F30S—DMD Study in MDX Mice Model

### 1. Study Objective

To examine the effects of N-163 Beta Glucan on MDX mice.

### 2. Experimental Design and Treatment Schedule

### 2.1. Study Groups

Group 1: Normal Fifteen C57BL/10SnSlc mice were without any treatment until sacrifice.
Group 2: Vehicle Fifteen MDX mice were orally administered vehicle [pure water] in a volume of 10 mL/kg once daily from Day 0 to 45.
Group 3: N-163 Beta Glucan Fifteen MDX mice were orally administered vehicle supplemented with N-163 Beta Glucan at a dose of 3 mg/kg as API in a volume of 10 mL/kg once daily from Day 0 to 45.

### 3. At Study Termination, Non-Fasting Blood was Collected Through Abdominal Vena Cava Using Precooled Syringes.

After sacrifice, quadriceps, gastrocnemius, soleus, plantaris, tibialis anterior, extensor digitorum longus, diaphragm, myocardium muscle were collected and frozen for analysis.

### Results

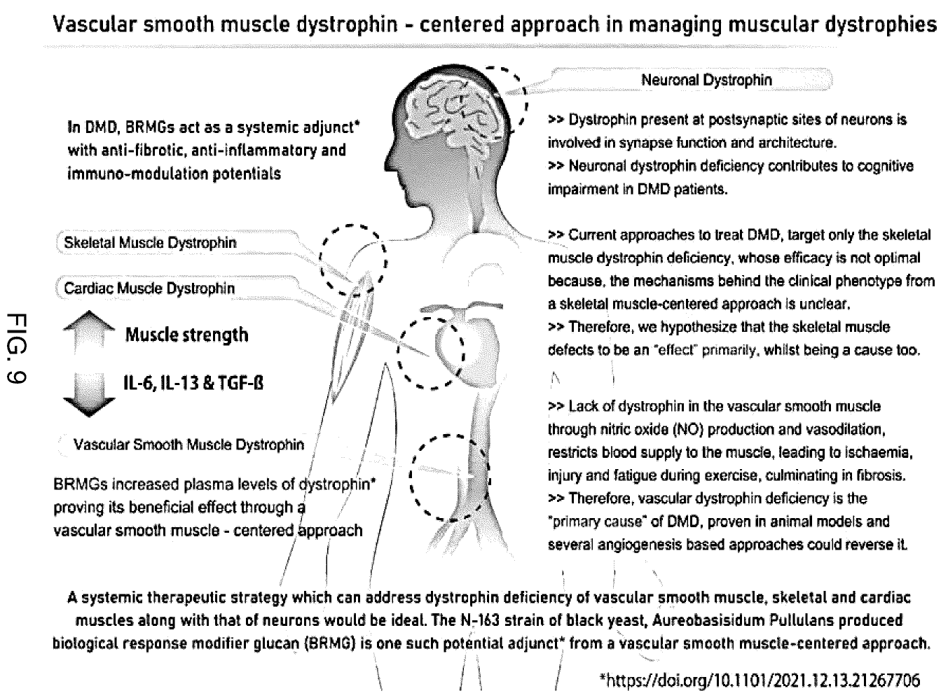

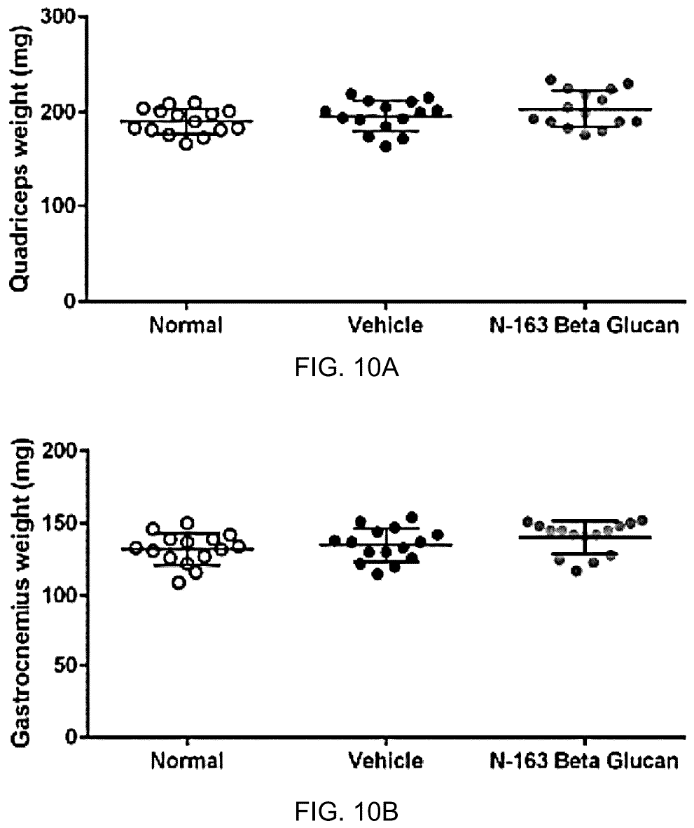

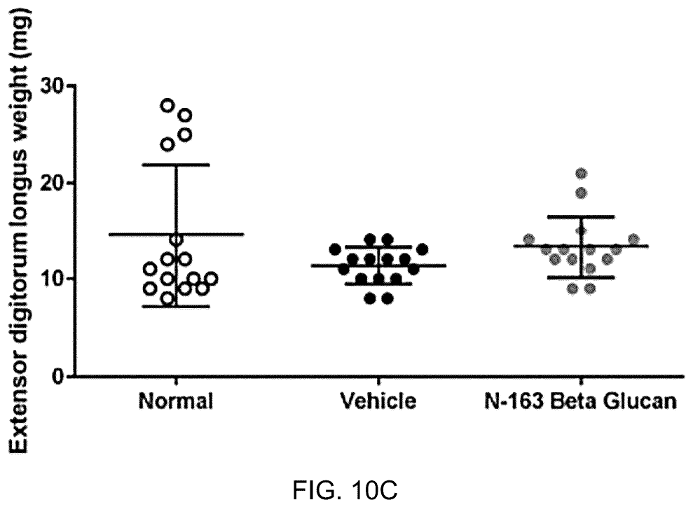

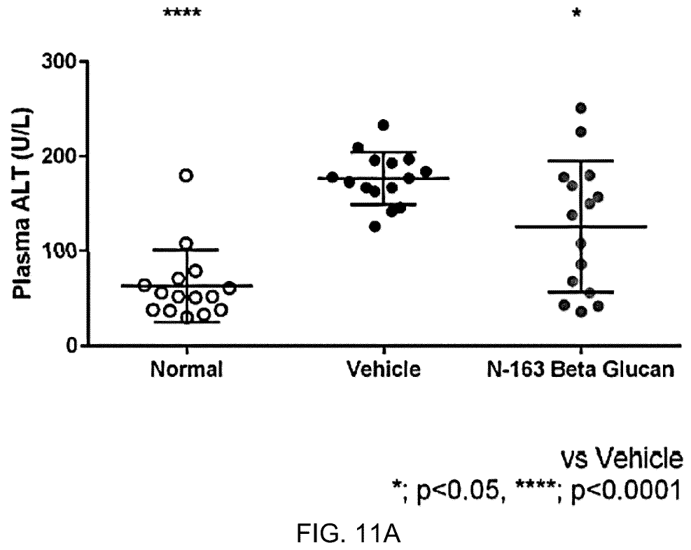

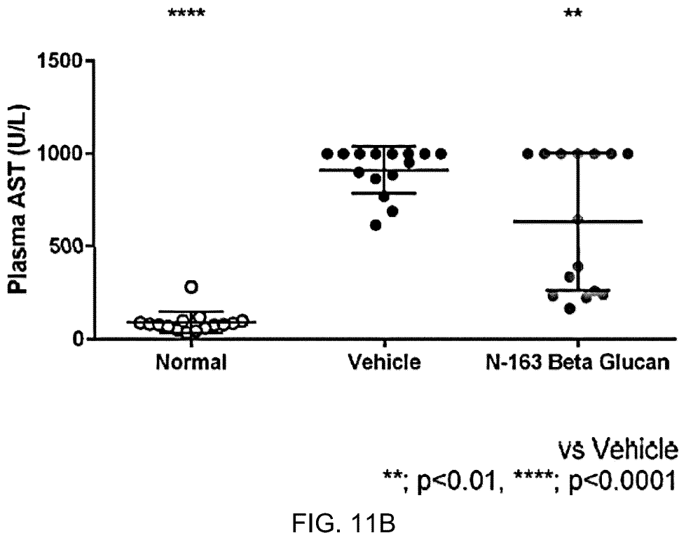

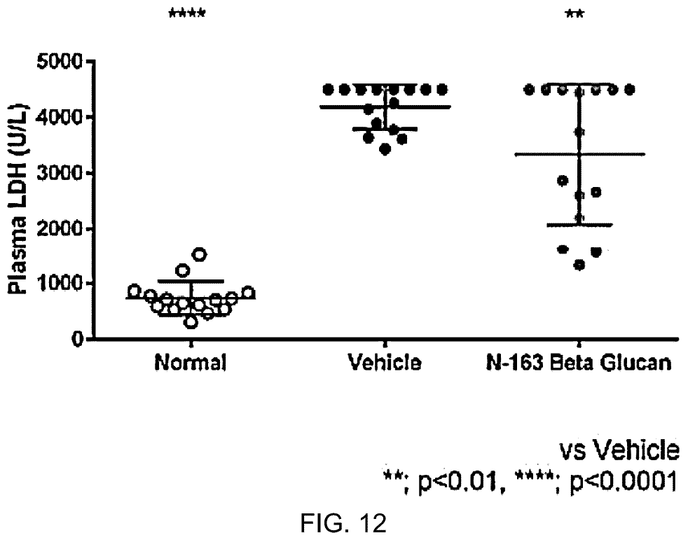

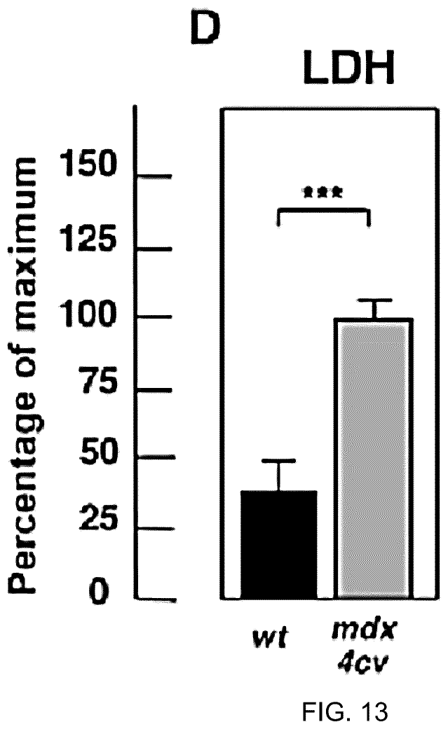

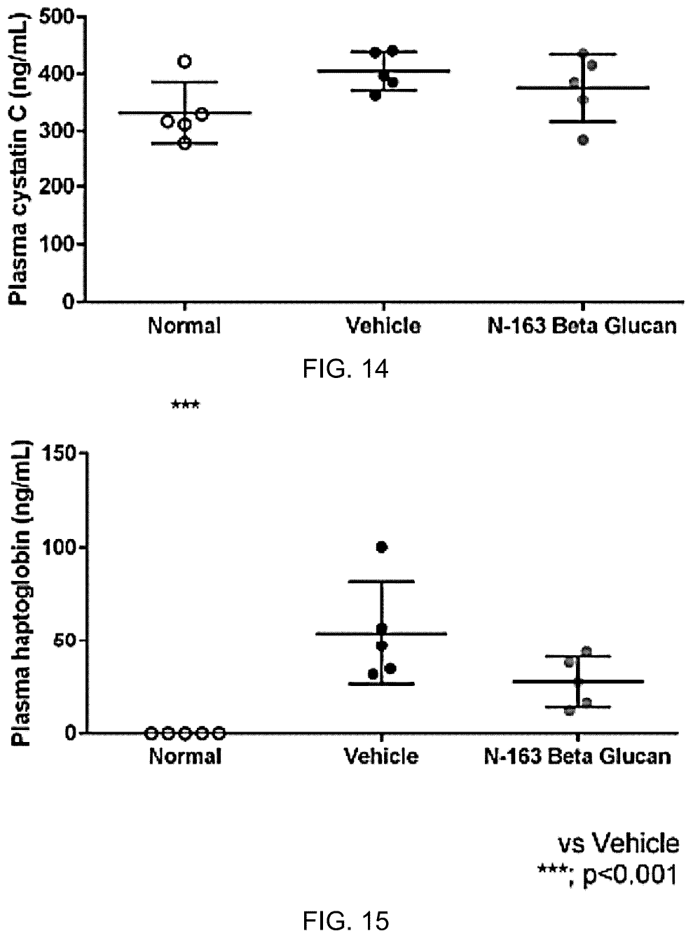

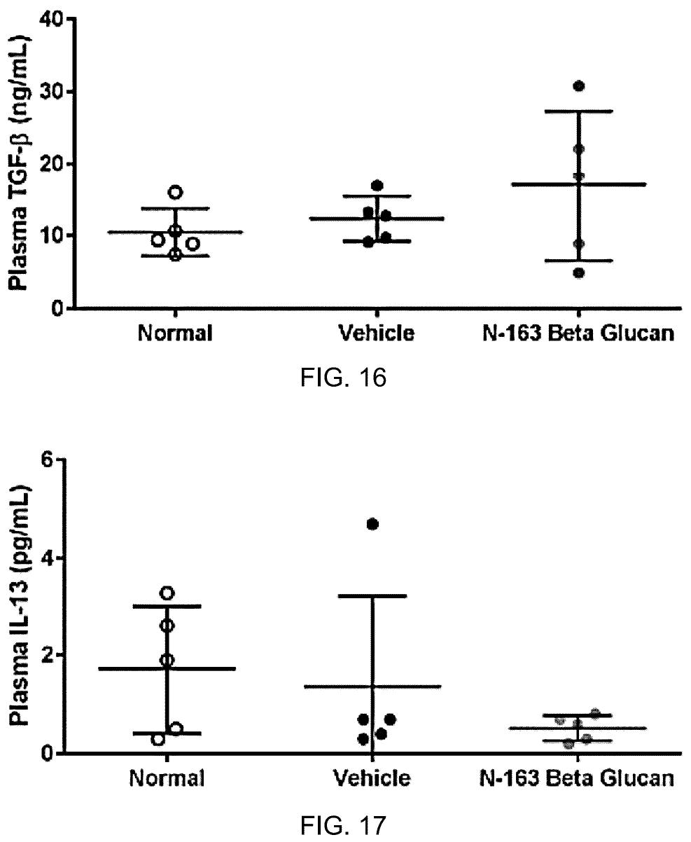

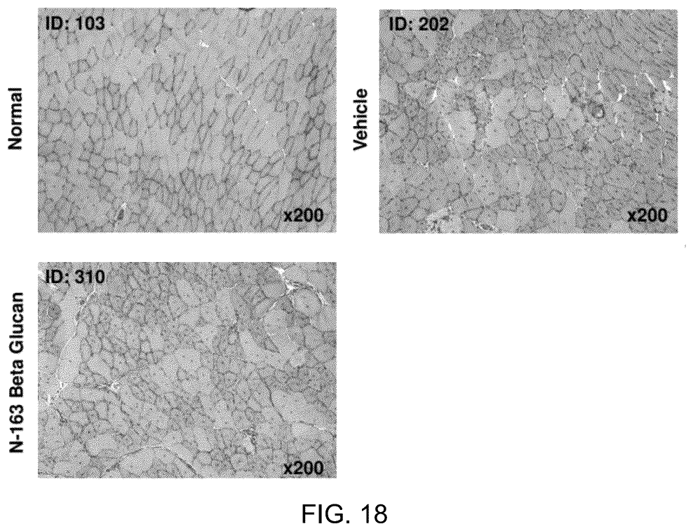

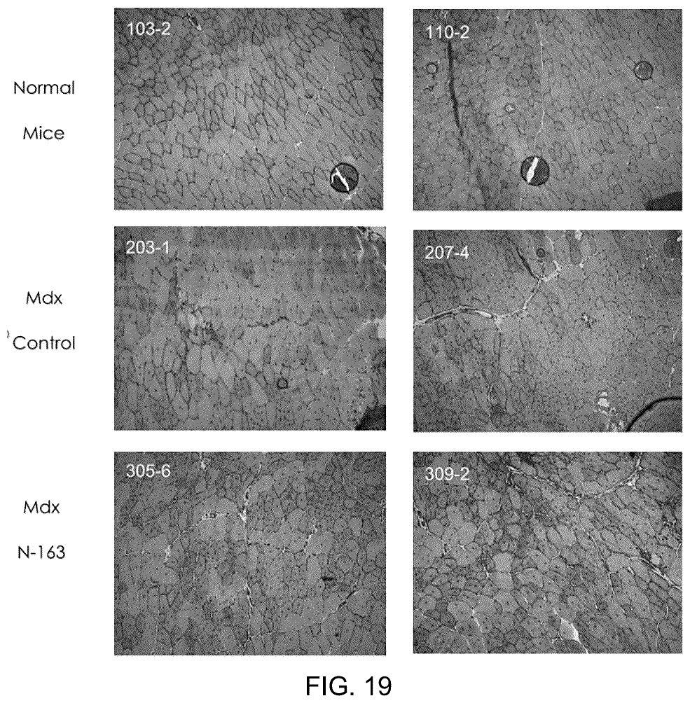

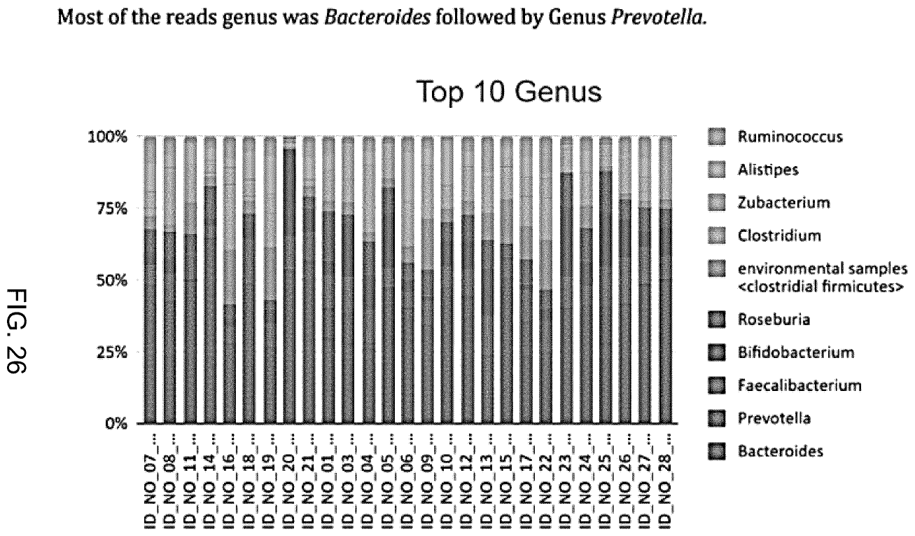

The results are shown in FIGS. 10-25. Increased in muscle weight in N-163 group is shown in FIG. 10. Decreased in plasma ALT and AST in N-163 group is shown in FIG. 11. Decrease in LDH in N-163 group is shown in FIG. 12. Decreased in Cystatin in N-163 group is shown in FIG. 14. Decrease in Haptoglobin in N-163 group is shown in FIG. 15. Increased in TGF-beta in N-163 group is shown in FIG. 16. Decrease in IL-13 in N-163 group is shown in FIG. 17. FIG. 19 and Table 3 shows Centro-Nucleated Fiber—Cell Count and images.

TABLE 3

| | | | Pheripheral | Central | Total cells |
|---|---|---|---|---|---|
| S. No | Group | Slide # | Nucleus | Nucleus | counted |
| 1 | Normal mice | 103-2 | 100 | 0 | 100 |
| Average | | | 97~100 | 0~3 | |
| 1 | Mdx | 201-3 | 27 | 73 | 100 |
| 2 | (Control) | 202-4 | 26 | 74 | 100 |
| 3 | | 203-2 | 12 | 88 | 100 |
| 4 | | 204-2 | 19 | 81 | 100 |
| 5 | | 205-1 | 14 | 86 | 100 |
| 6 | | 206-5 | 26 | 74 | 100 |
| 7 | | 207-4 | 20 | 80 | 100 |
| 8 | | 208-2 | 15 | 85 | 100 |
| 9 | | 209-2 | 22 | 78 | 100 |
| 10 | | 210-2 | 19 | 81 | 100 |
| Average | | | 20 | 80 | |
| 1 | Mdx | 301-2 | 18 | 82 | 100 |
| 2 | (N-163) | 302-2 | 28 | 72 | 100 |
| 3 | | 303-2 | 24 | 76 | 100 |
| 4 | | 304-6 | 27 | 73 | 100 |
| 5 | | 305-6 | 22 | 78 | 100 |
| 6 | | 306-3 | 24 | 76 | 100 |
| 7 | | 307-1 | 10 | 90 | 100 |
| 8 | | 308-1 | 30 | 70 | 100 |
| 9 | | 309-2 | 24 | 76 | 100 |
| 10 | | 310-4 | 25 | 75 | 100 |
| Average | | | 23.2 | 76.8 | |

The percentage of centrally nucleated fibres (CNF) evaluated by Masson's Trichrome staining was 0 in the normal group while it increased to 80% in the vehicle group and it was 76.8% in N-163 group.

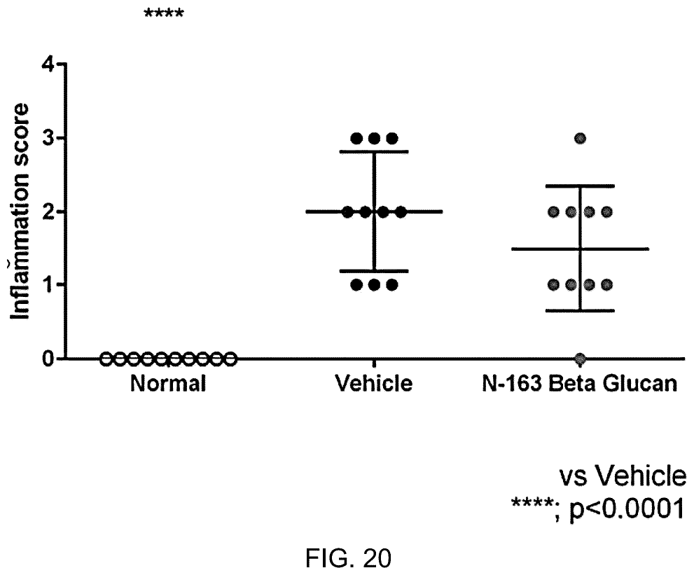

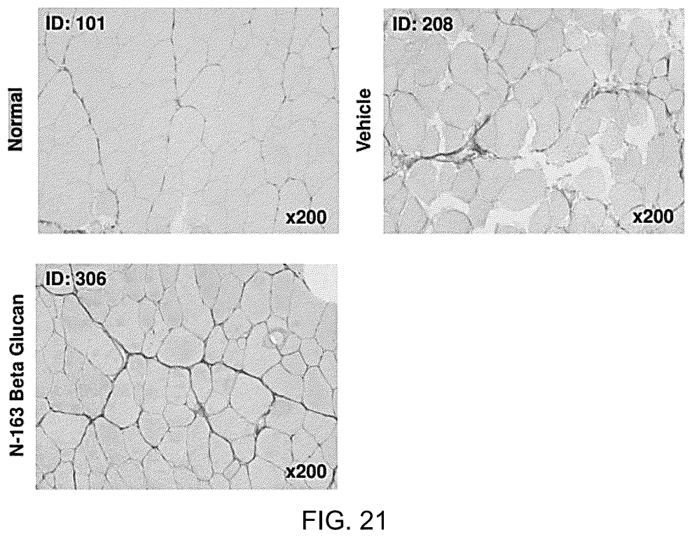

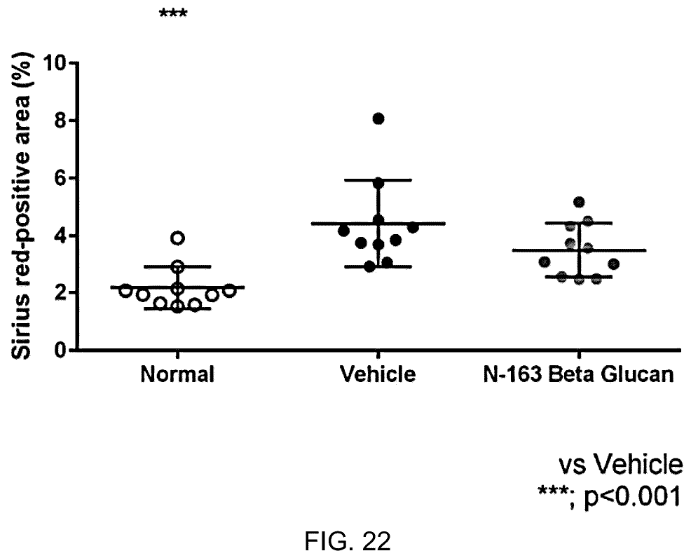

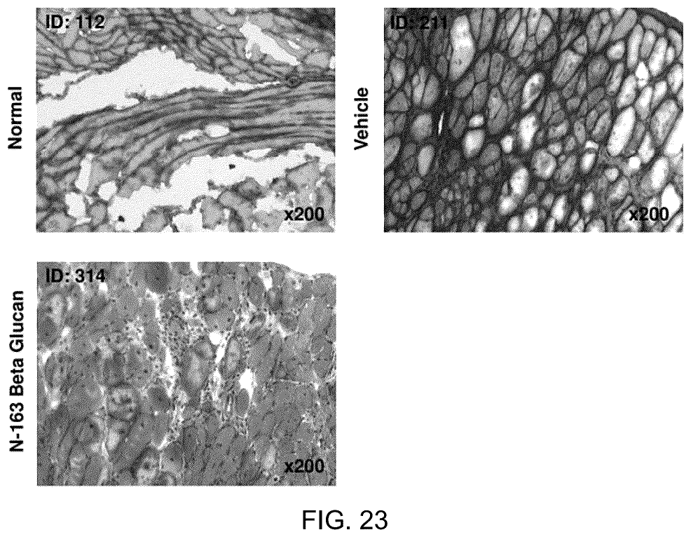

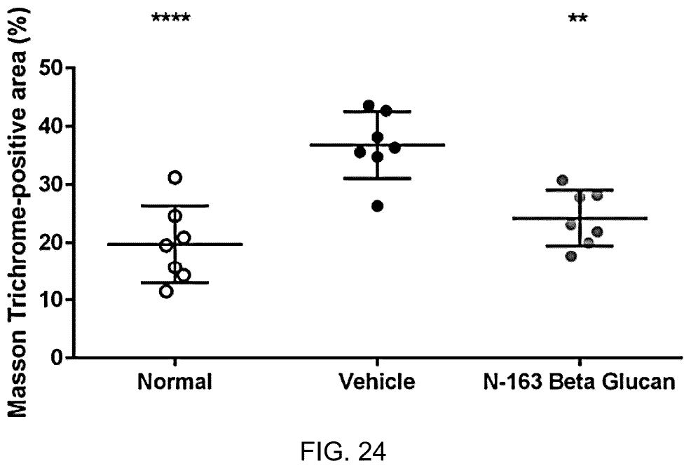

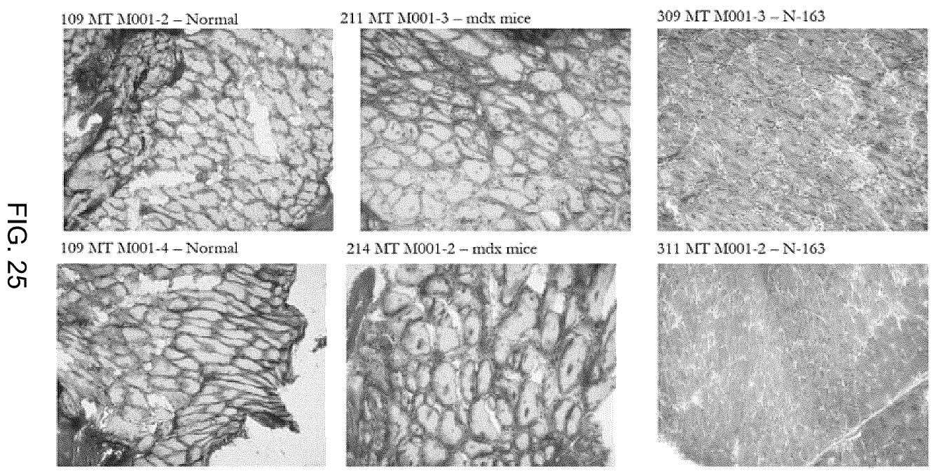

Decreased Inflammation Score in N-163 is shown in FIG. 20. Decreased fibrosis score in N-163 are shown in FIGS. 21 and 22 (sirius red staining). FIGS. 23 and 24 show that fibrosis are decreased in N-163 (Masson's Trichrome staining).

In our study the CNF increased in mdx mice compared to normal mice due to increase in necrotic fibres. The number of CNF decreased after N-163 as the necrosis is tackled by N-163 by reduction in inflammation and the number of peripheral nucleated fibres increased after N-163 showing that normal dystrophin positive fibers that are matured are increased after N-163 administration.

The mdx mice had larger sized cells and increased fibrosis compared to normal mice while after N-163 treatment, the cells resembled the normal cells and the fibrosis also decreased.

### Discussion

Increased in lipopolysaccharides leads to chronic inflammation and treatment with Adiponectin (ApN) has been able to provide therapeutic benefits in DMD through insulin-sensitizing, fat-burning, and anti-inflammatory/antioxidative stress properties. In the present study, supplementation with N-163 BRMG has been able to decrease the LDH level which would be helpful in alleviating chronic inflammation [Am J Pathol. 2017 July; 187(7):1577-1585.]. Dramatic elevation in urinary titin excretion has been reported in DMD patients and in dystrophin deficient rodents, coincident with the development of systemic skeletal muscle damage [Neuromuscul Disord. 2017 July; 27(7):635-645.]. In the present study, there was significant decrease in urine titin levels after N-163 administration, therefore indicating resolution of skeletal muscle damage. The inducible plasma marker haptoglobin is an acute phase response protein which is secreted in relation to tissue damage and sterile inflammation and has been reported to be elevated in DMD mice. In the current study, there was significant decrease in plasma marker haptoglobin after N-163 administration [Int J Mol Med. 2017 June; 39(6):1357-1370.]. Although TGF-β has been reported to be involved in fibrosis, the studies show that TGF-β also functions as an anti-inflammatory cytokine and it helps balance between inflammation and fibrosis [Arterioscler Thromb Vasc Biol. 2002 Jun. 1; 22(6):975-82.]. Treatment with steroid and vitamin-D in a study has been shown to decrease IL-13 which is a profibrotic marker [Cytokine. 2018 February; 102:55-61.]. In the current study, the N-163 BRMG has been shown to decrease IL-13. In the present study TGF-β increased in N-163 group. The N-163 Beta Glucan group showed a significant decrease in the fibrosis area (Masson's Trichrome-positive area) compared with the Vehicle group. Inflammation score and fibrosis area in the N-163 Beta Glucan group tended to decrease compared with the Vehicle group. In DMD, Sporadic dystrophin-positive muscle fibres, called revertant fibres (RFs) are thought to arise from skeletal muscle precursor cells and clonally expand with age due to the frequent regeneration of necrotic fibres [Sci Rep. 2016 Dec. 7; 6:38371.]. The nuclei of newly regenerated muscle fibres are centrally located while those of mature muscle fibres are peripherally located. In our study the CNF increased in mdx mice compared to normal mice due to increase in necrotic fibres. The number of CNF decreased after N-163 as we speculate that the necrosis is tackled by N-163 by reduction in inflammation and the number of peripheral nucleated fibres increased after N-163 showing that normal dystrophin positive fibres that are matured are also increased after N-163 administration. The N-163 group showed a significant decrease in the fibrosis area (Masson's Trichrome-positive area) compared with the Vehicle group. Inflammation score and fibrosis area in the N-163 Beta Glucan group tended to decrease compared with the Vehicle group.

### Example 4: F16S—DMD—Human Study

### Methods

Twenty-seven patients with Duchenne muscular dystrophy (DMD)—nine in the control arm (undergoing conventional therapies)—participated and 18 in the treatment arm (N-163 beta glucan supplement along with conventional therapies).

They participated in the study for 45 days.

Fecal samples were collected at baseline and after 45 days and subjected to whole genome metagenome sequencing for gut microbiome analysis.

### Results

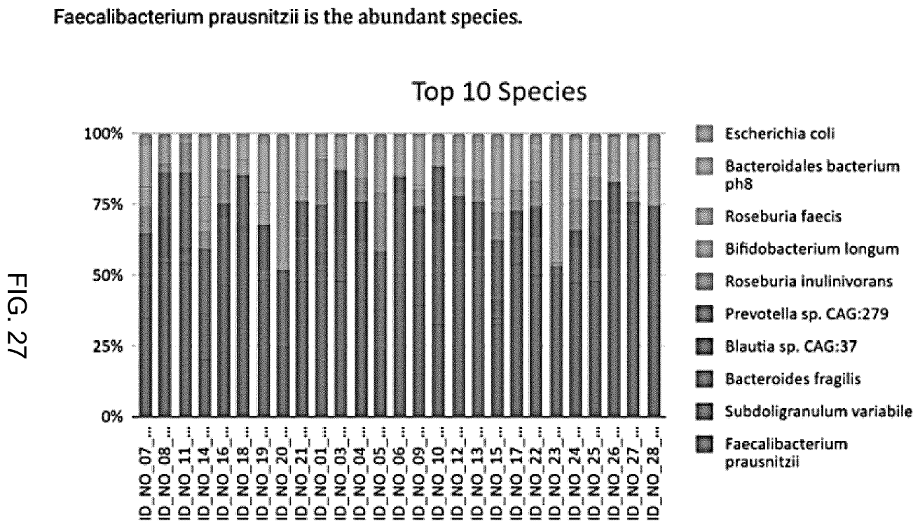

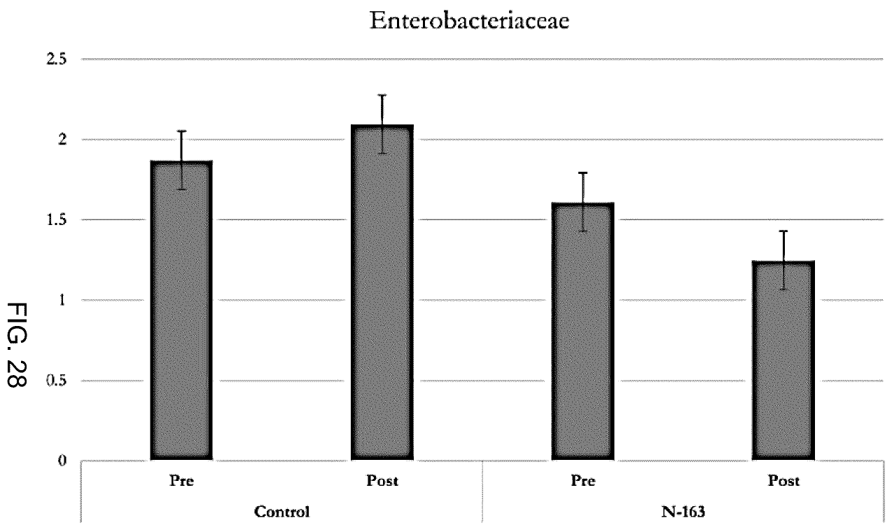

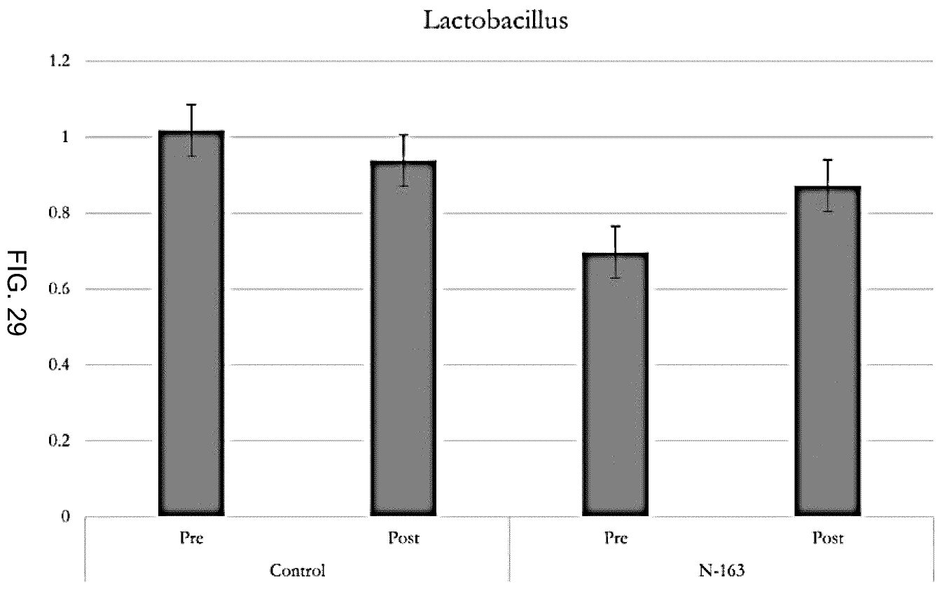

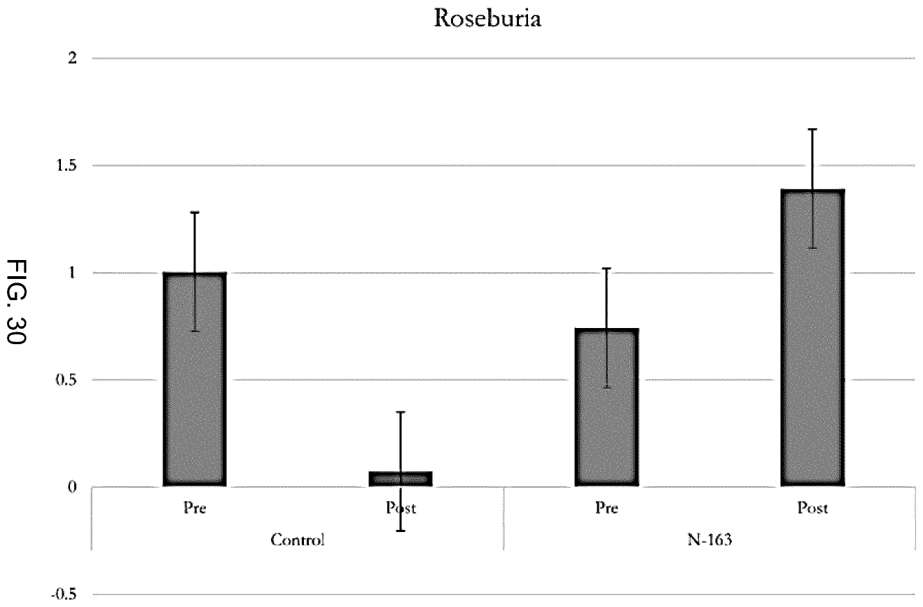

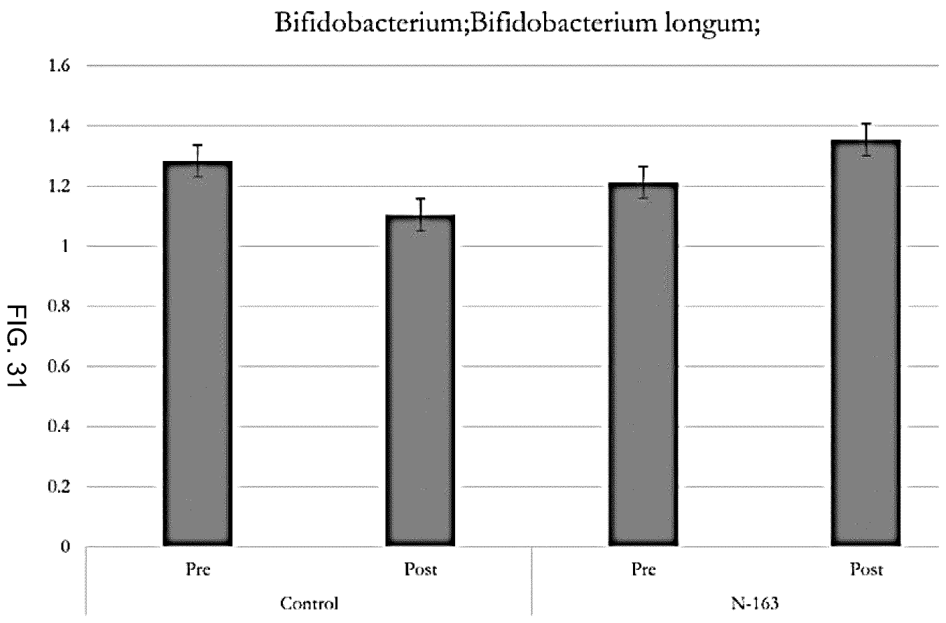

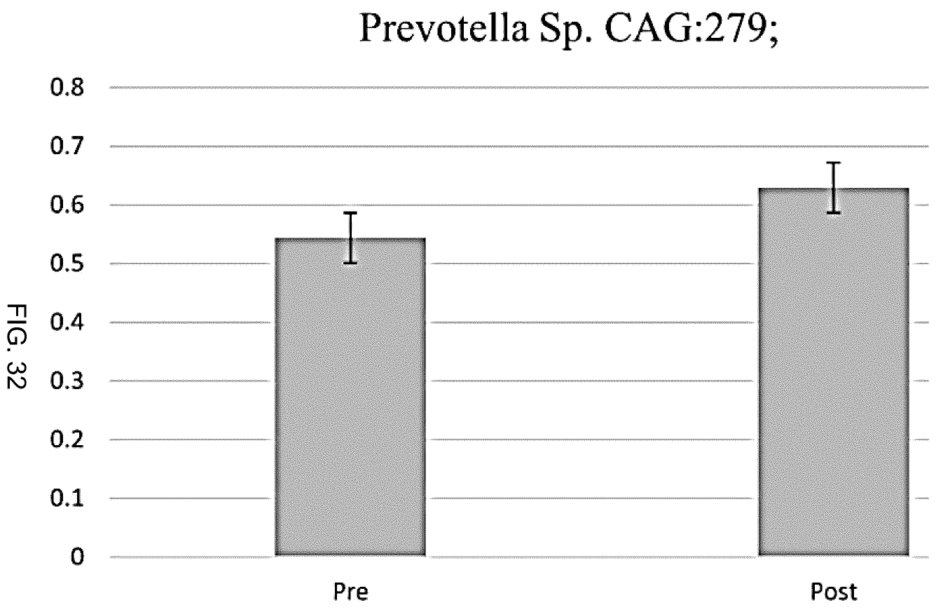

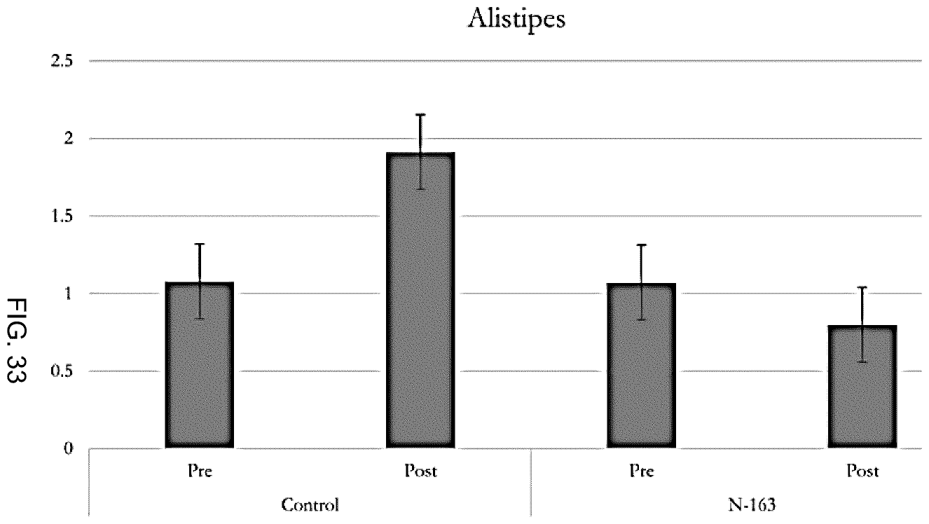

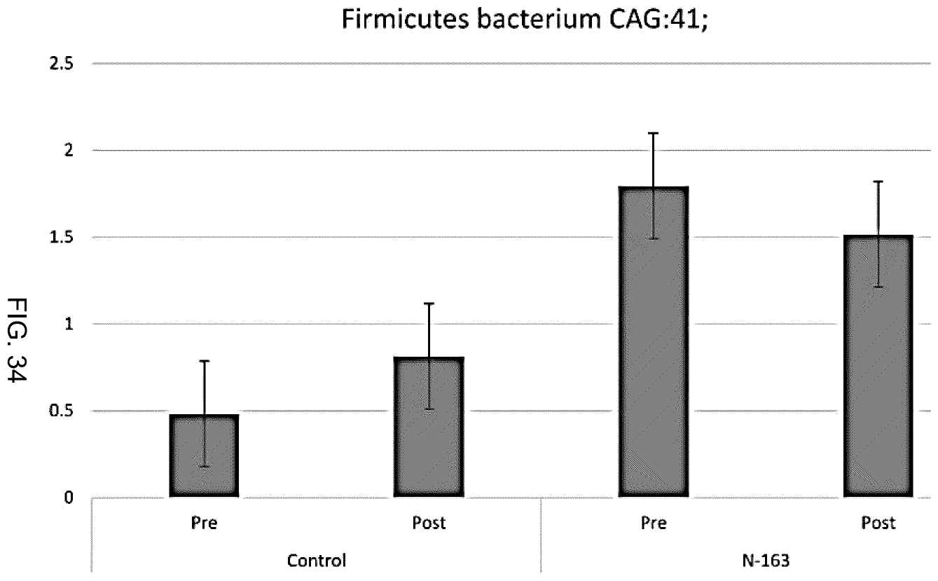

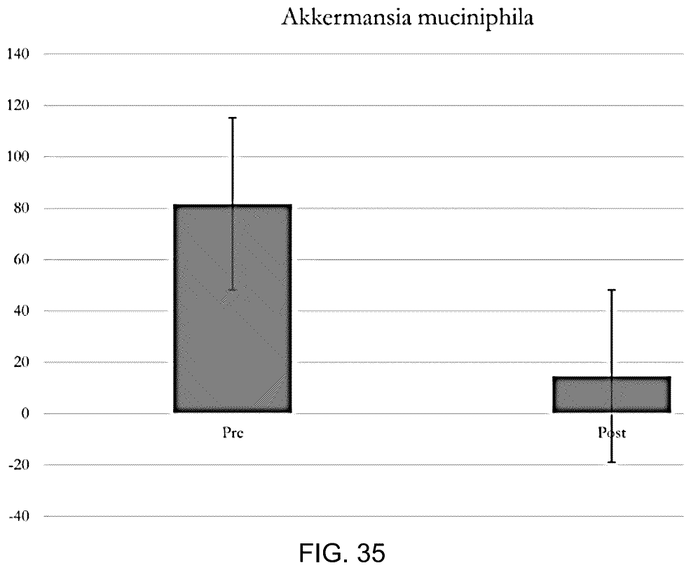

The results are shown in FIGS. 26-35. Increase in Bacteroides after N-163 is shown in FIG. 26. FIG. 27 shows that Faecalibacterium prausnitzii was most abundant species after N-163. Decrease in Enterobacteriaceae after N-163 beta glucan but increase in control group is shown in FIG. 28. Increase in Lactobacillus after N-163 beta glucan but decrease in control group is shown in FIG. 29. Increase in Roseburia after N-163 beta glucan but decrease in control group is shown in FIG. 30. Increase in Bifidobacterium after N-163 beta glucan but decrease in control group is shown in FIG. 31. Increase in Prevotella after N-163 beta glucan is shown in FIG. 32. Decrease in Alistipes after N-163 beta glucan but increase in control group is shown in FIG. 33. Decrease in Firmicutes after N-163 beta glucan but increase in control group is shown in FIG. 34. Decrease in Akkermansia muciniphila after N-163 beta glucan is shown in FIG. 35.

### Discussion

Enterobacteriaceae, has been described as enhancing the inflammatory response and therefore its decrease will be beneficial in multiple sclerosis and DMD (Ref: https://www.mdpi.com/2076-2607/9/4/697/htm).

Restoration of Lactobacillus species has been shown to decrease inflammatory cytokines and its increase by N-163 is beneficial in MS (Ref: https://www.mdpi.com/2076-2607/9/4/697/htm).

Increase in Roseburia is a marker of intestinal health as it is a butyrate (a beneficial metabolite) producing bacteria (Ref: Future Microbiol 2017: 157-170).

Bifidobacterium longum has been reported to increase both muscle function and cognitive ability (Ref: https://www.ncbi.nlm.nih.gov/pmc/articles/PMC7745561/). The increase in Bifidobacterium longum after N-163 is therefore beneficial.

Alistipes contributes to inflammation and epithelium alterations (https://www.ncbi.nlm.nih.gov/pmc/articles/PMC7745561/). Firmicutes, play a role in the onset of depression via affecting the inflammation levels of host (https://www.frontiersin.org/articles/10.3389/fcimb.2022.831186/). Increase in the abundance of Akkermansia has been reported in patients with PD and MS (https://pubmed.ncbi.nlm.nih.gov/28843021/). Therefore the decrease of these bacteria is beneficial.

### Example 5: F16S—DMD Six Months Human Study

### Long DMD Study

Clinical Study was conducted to Evaluate the Anti-Inflammatory and Beneficial Effects of N-163 Beta Glucan food supplement on Duchenne muscular dystrophy (DMD) Patients for 6 months.

Patients aged between 3-30 years were included in the study.

Twenty-six patients have been enrolled and the study is ongoing.

### Results

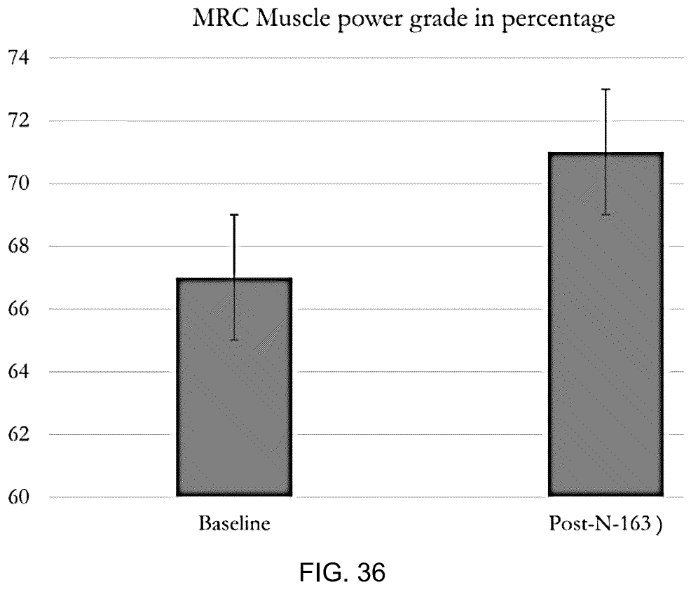

The result is shown in FIG. 36. Increase in muscle power is shown in FIG. 36. MRC—Muscle power increased from an average of 67% to 71% in the two months interim report.

### Discussion

An increase in muscle strength has been reported during the first six months of treatment, followed by a stabilisation period of two years, with a subsequent decline that is slower than in the untreated patients in the case of conventional therapies such as steroids (Cochrane Database Syst Rev. 2008; 1:CD003725-CD003725.) which have side effects. In the present study, after a safe food supplement N-163, an increase in muscle power has been reported after the first two months of treatment itself.

### Example 6: F32S—LGMD—Human Study

### LGMD Study

A Clinical Study was conducted to evaluate the Anti-Inflammatory and Beneficial Effects of N-163 food supplement on another Muscular Dystrophy other than DMD Patients for 60 days.

Patients aged between 3-70 years were included in the study.

Six patients completed the study.

### Results

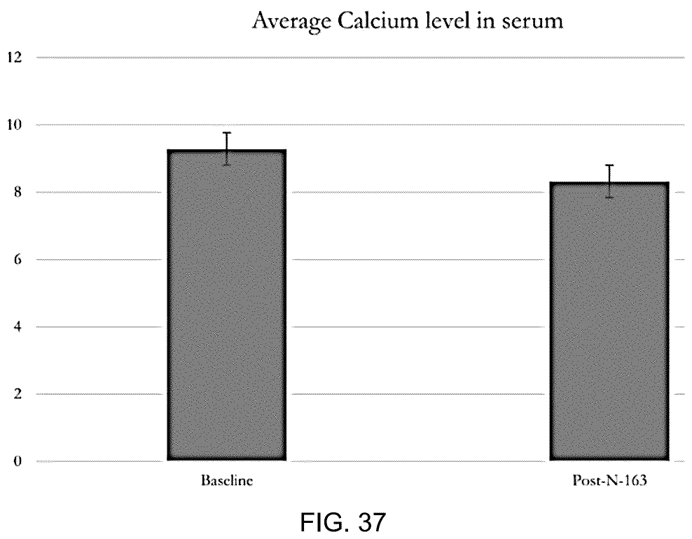

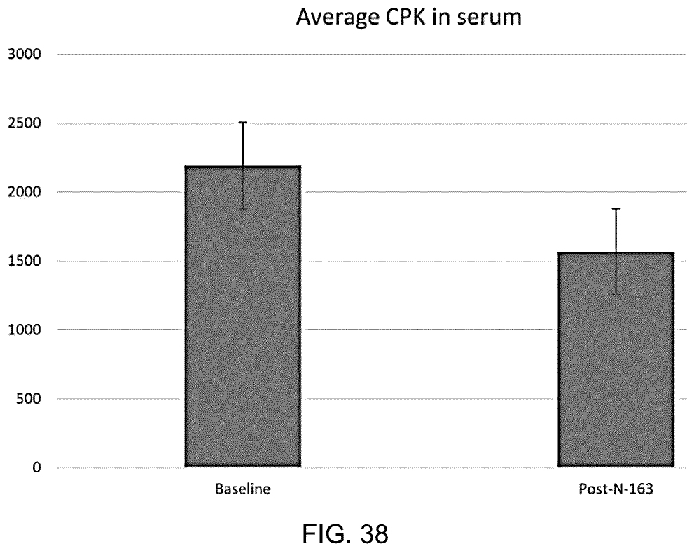

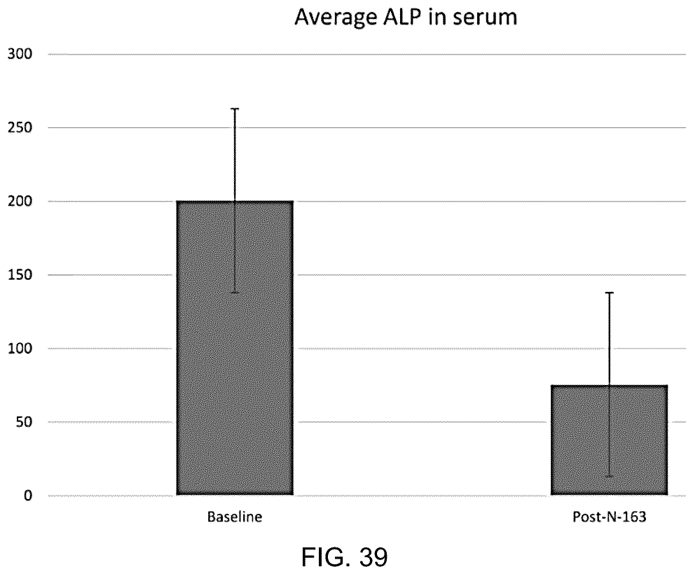

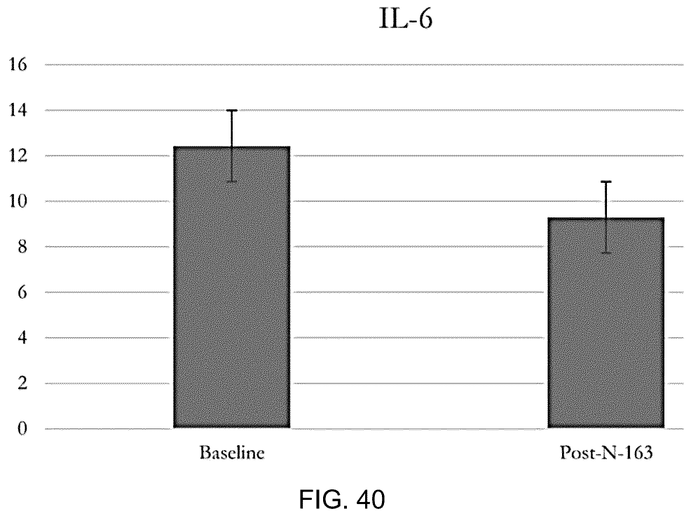

The results are shown in FIGS. 37-39. Decreased calcium in serum is shown in FIG. 37. Decreased CPK in serum after N-163 is shown in FIG. 38. Decreased ALP in serum after N-163 is shown in FIG. 39.

### Discussion

Abnormal levels of calcium have been reported in muscular dystrophies (Mareedu et al. Front.Physio. 2021). Elevated CPK levels are indicative of muscle disease (https://emedicine.medscape.com/article/1259041-workup#:˜:text=Early %20in %20the %20disease %20process,elevation %20noted %20in%20Becker %20MD.). LGMD is characterized by increased ALT, AST and LDH (Dis.Markers 2015; 2015:543282). Therefore, the decrease of these markers after N-163 is beneficial.

### Example 7: F27S—Multiple Sclerosis-Human Study

### Methods

An Open Label, Prospective, Non-Randomised, Non-Comparative Single Arm Clinical Study to Evaluate the Effects of N163 Strain of Aureobasidium Pullulans Produced p 1,3-16 Glucans (Beta Glucan) in modulating the immunity in patients with Multiple Sclerosis is being conducted.

Study period=2 months.
Seven patients have completed the study.

### Results

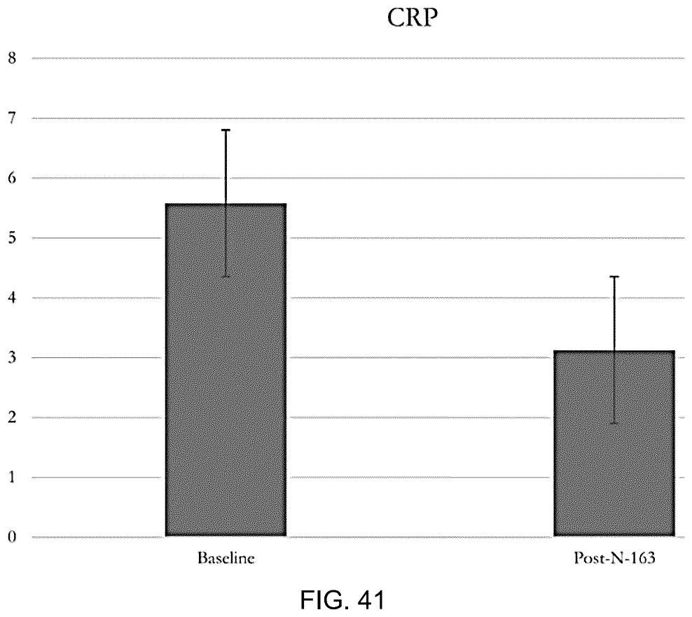

The results are shown in FIGS. 40 and 41. Decrease in IL-6 after N-163 is shown in FIG. 40. Decrease in C-reactive protein (CRP) after N-163 is shown in FIG. 41.

### Discussion

IL-6 and CRP which are markers of inflammation has been shown to be increased in MS and DMD, driving the disease pathogenesis (Biomed Res Int. 2015; 2015; 891972). Blockade of IL-6 has been reported as a therapy for muscular dystrophies (Ebiomedicine 2015; 2(4)). Therefore, the decrease of these markers after N-163 is beneficial.

### Example 8: F27S—Multiple Sclerosis Gut Microbiome Analysis

Methods of fecal microbiota metagenome sequencing of MS patients:
Samples were sequenced using Novaseq V1.5 with a read length of 151 bp.
The sample were taken for whole genome metagenome analysis. Initially, the reads were filtered for human DNA contamination. The alignment to human genome was around 0.01%-1.6%. The filtered reads were further used downstream analysis.
Also, de novo assembly was carried out using the pre-processed reads to obtain the scaffolds.
These scaffolds were then used for gene prediction.

### Results

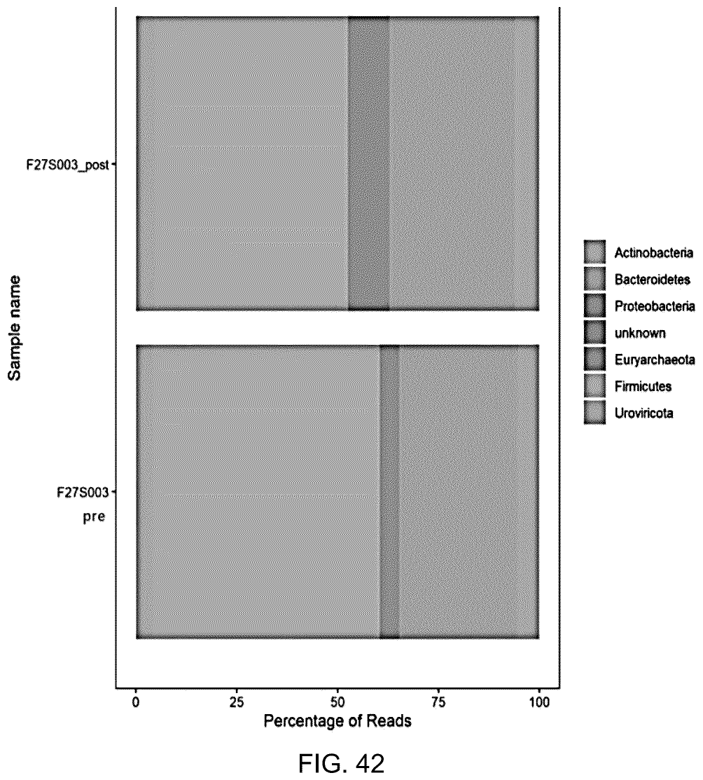

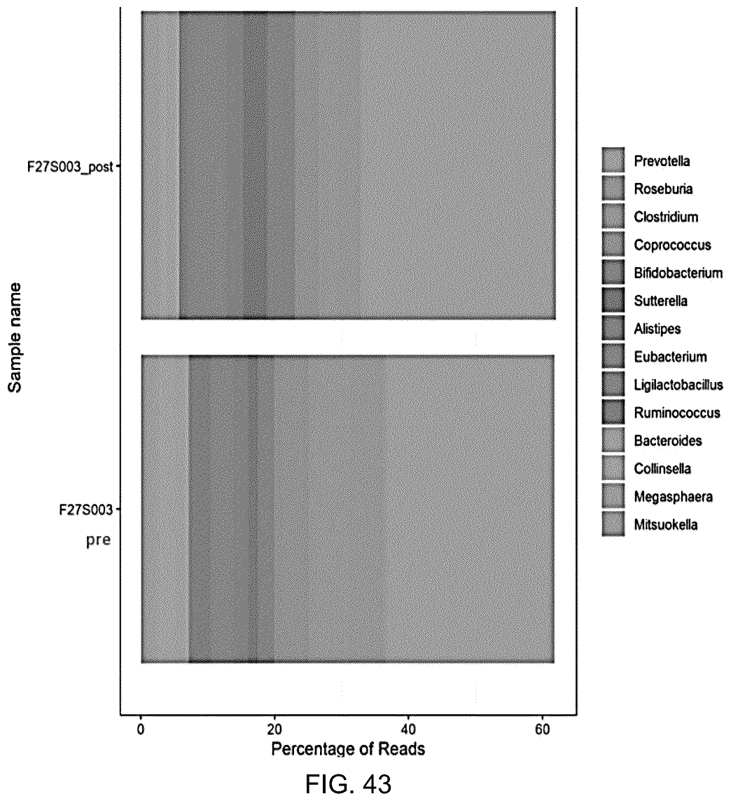

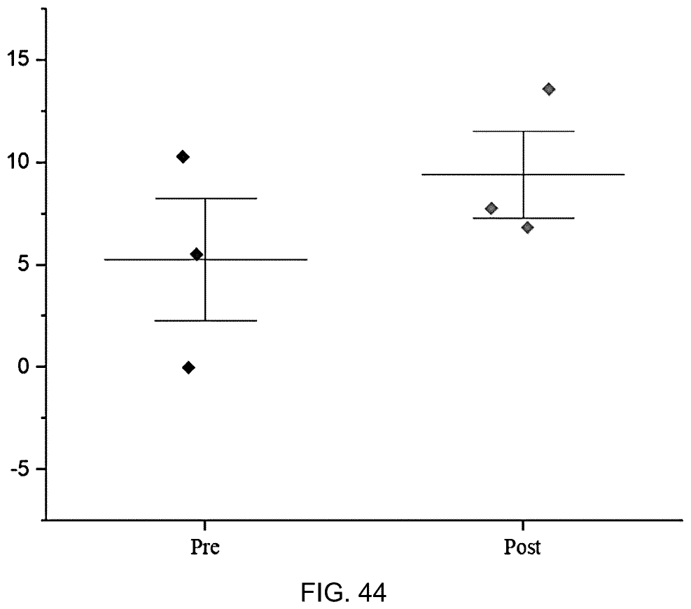

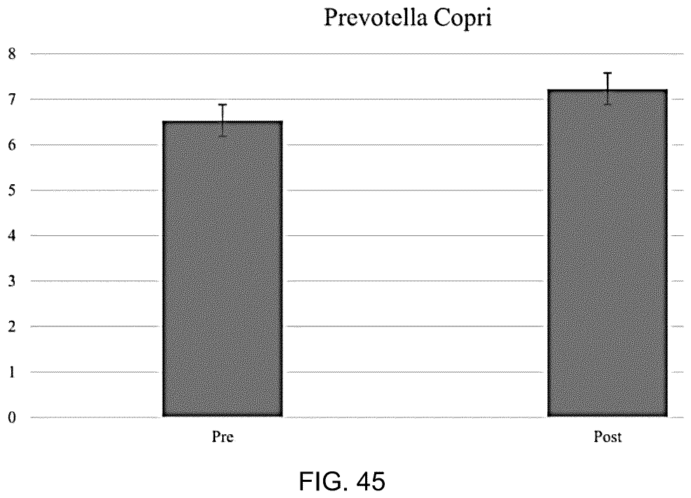

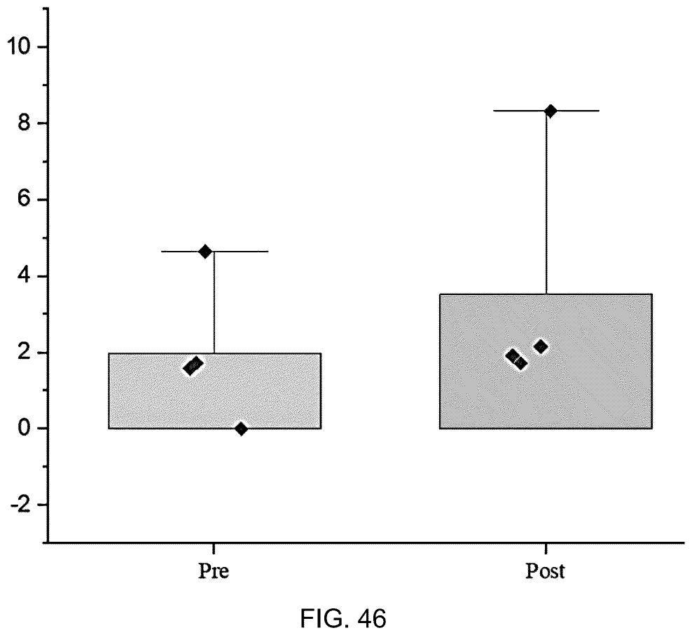

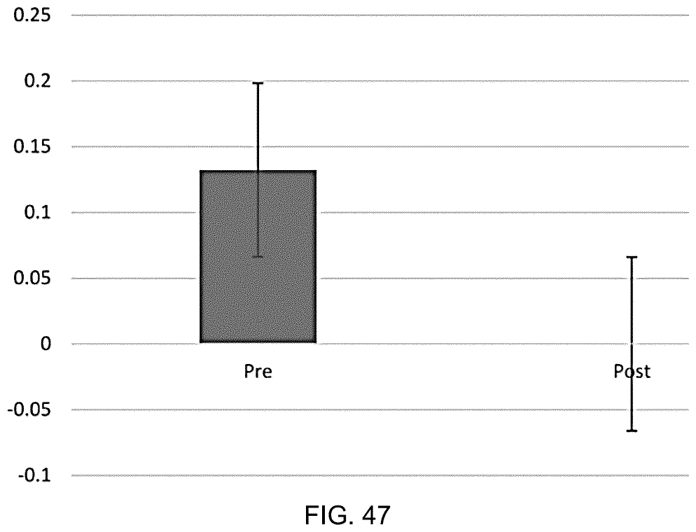

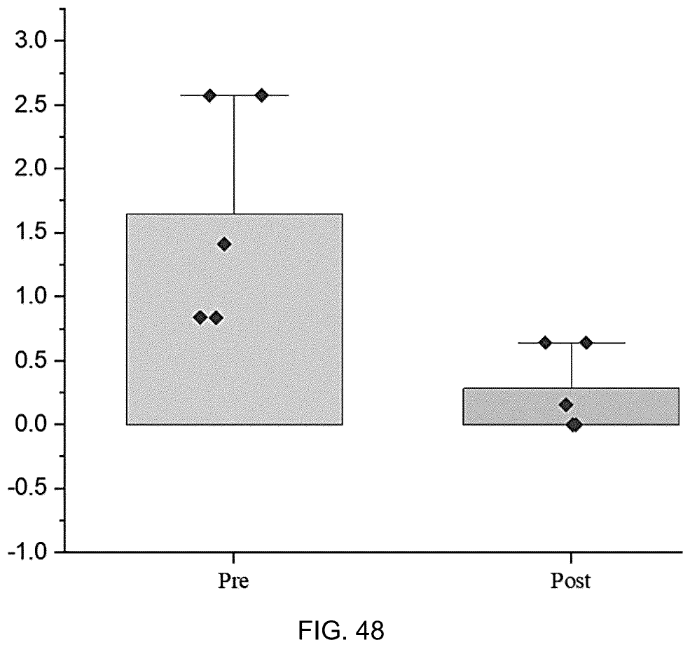

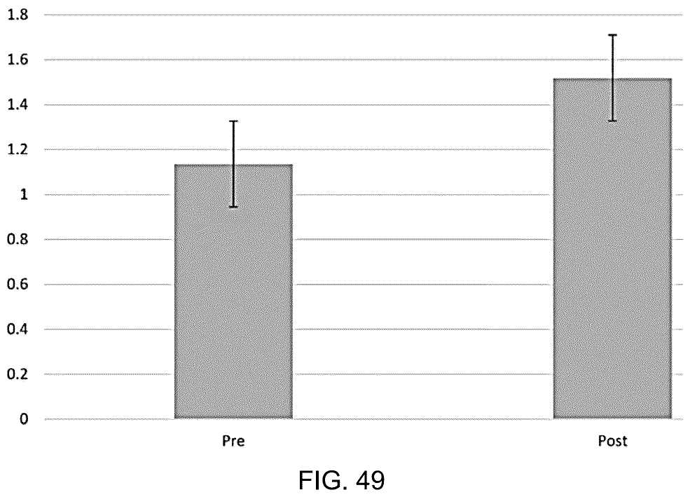

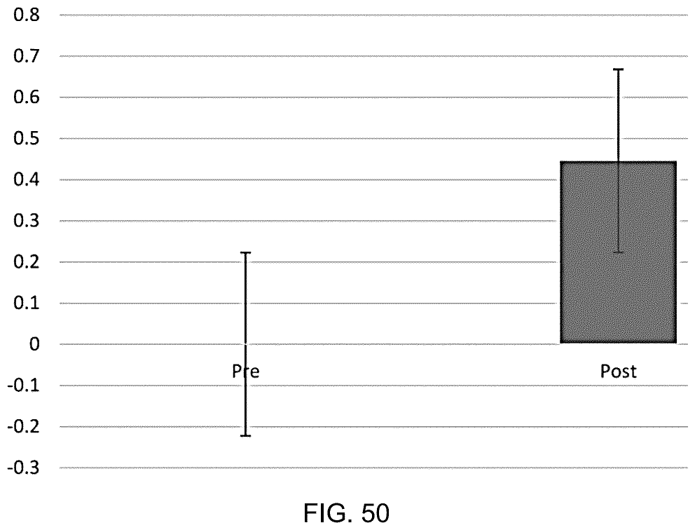

The results are shown in FIGS. 42-50. Increase in Bacteroides and decrease in firmicutes after N-163 is shown in FIG. 42. Increase in Prevotella after N-163 is shown in FIG. 43. FIG. 44 shows that Faecalibacterium prausnitzii abundance increased post-N-163 intervention. FIG. 45 shows that Bifidobacterium longum abundance increased post-N-163 intervention. FIG. 46 shows that Bifidobacterium longum abundance increased post-N-163 intervention. FIG. 47 shows that Streptococcus parasanguinis abundance decreased post-N-163 intervention. FIG. 48 shows that Streptococcus salivarius abundance decreased post-N-163 intervention. FIG. 49 shows that Parabacteroides distasonis abundance increased post-N-163 intervention. FIG. 50 shows that Roseburia intestinalis abundance increased post-N-163 intervention.

### Discussion

MS patients have presented gut dysbiosis with a reduction in bacteria belonging to the Prevotella genus especially Prevotella copri (https://pubmed.ncbi.nlm.nih.gov/30513004/; https://www.ncbi.nlm.nih.gov/pmc/articles/PMC5730390/). Fecalibacterium which is an anti-inflammatory commensal bacterium has been reported to be low in patients with MS (J Investig Med. 2015 June; 63(5):729-34. doi: 10.1097/JIM.0000000000000192.). Bifidobacteria has substantial roles in regulation of immune response and lower frequency of bifidobacteria in gut of MS patients has been reported (https://www.sciencedirect.com/science/article/abs/pii/S2211034819303554) Parabacteroides distasonis to be reduced in abundance in the MS patients (https://www.pnas.org/doi/10.1073/pnas.1711235114). R. intestinalis has been shown to prevent intestinal inflammation (https://www.frontiersin.org/articles/10.3389/fcimb.2021.757718/full). Therefore, the increase of these bacteria after N-163 is beneficial.

Streptococcus parasanguinis (www.pnas.org/cgi/doi/10.1073/pnas.2011703117), S. salivarius/thermophilus (www.pnas.org/cgi/doi/10.1073/pnas.2011703117) has been reported to be significantly increased in MS patients. Therefore, the decrease of these bacteria after N-163 is beneficial.

### Modifications and Other Embodiments

Various modifications and variations of the described glucan products, compositions and methods as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the chemical, biological, medical, environmental, cosmetic or food arts or related fields are intended to be within the scope of the following claims.

### REFERENCES

1. Theadom A, Rodrigues M, Roxburgh R, Balalla S, Higgins C, Bhattacharjee R, Jones K, Krishnamurthi R, Feigin V. Prevalence of muscular dystrophies: a systematic literature review. Neuroepidemiology. 2014; 43(3-4):259-68. doi: 10.1159/000369343.
2. Nagy S, Hafner P, Schmidt S, Rubino-Nacht D, Schadelin S, Bieri O, Fischer D. Tamoxifen in Duchenne muscular dystrophy (TAMDMD): study protocol for a multicenter, randomized, placebo-controlled, double-blind phase 3 trial. Trials. 2019 Nov. 21; 20(1):637. doi: 10.1186/s13063-019-3740-6. PMID: 31752977; PMCID: PMC6869203.
3. Rosenberg A S, Puig M, Nagaraju K, Hoffman E P, Villalta S A, Rao V A, Wakefield L M, Woodcock J. Immune-mediated pathology in Duchenne muscular dystrophy. Sci Transl Med. 2015 Aug. 5; 7(299):299rv4. doi: 10.1126/scitranslmed.aaa7322. PMID: 26246170; PMCID: PMC5951380.
4. Manzur A Y, Kuntzer T, Pike M, Swan A. Glucocorticoid corticosteroids for Duchenne muscular dystrophy. Cochrane Database Syst Rev. 2004(2):CD003725. doi: 10.1002/14651858
5. https://www.niams.nih.gov/newsroom/spotlight-on-research/optimizing-steroid-treatment-duchenne-muscular-dystrophy
6. Licitra R, Marchese M, Brogi L, Fronte B, Pitto L, Santorelli F M. Nutraceutical Screening in a Zebrafish Model of Muscular Dystrophy: Gingerol as a Possible Food Aid. Nutrients. 2021 Mar. 19; 13(3):998.
7. Ikewaki N, Sonoda T, Kurosawa G, Iwasaki M, Dedeepiya V D, Senthilkumar R, Preethy S, Abraham S J K. Immune and metabolic beneficial effects of Beta 1,3-1,6 glucans produced by two novel strains of Aureobasidium pullulans in healthy middle-aged Japanese men: An exploratory study. medRxiv 2021.08.05.21261640; doi: 10.1101/2021.08.05.21261640
8. Ikewaki N, Onaka T, Ikeue Y, Nagataki M, Kurosawa G, Dedeepiya V D, Rajmohan M, Vaddi S, Senthilkumar R, Preethy S, Abraham S J K. Beneficial effects of the AFO-202 and N-163 strains of Aureobasidium pullulans produced 1,3-1,6 beta glucans on non-esterified fatty acid levels in obese diabetic KKAy mice: A comparative study.bioRxiv 2021.07.22.453362; doi: 10.1101/2021.07.22.453362
9. Ikewaki N, Kurosawa G, Iwasaki M, Preethy S, Dedeepiya V D, Vaddi S, Senthilkumar R, Levy G A, Abraham S J K. Hepatoprotective effects of Aureobasidium pullulans derived Beta 1,3-1,6 biological response modifier glucans in a STAM-animal model of non-alcoholic steatohepatitis. bioRxiv 2021.07.08.451700; doi: 10.1101/2021.07.08.451700
10. https://mrc.ukri.org/research/facilities-and-resources-for-researchers/mrc-scales/mrc-muscle-scale/
11. McDonald C M, Henricson E K, Han J J, Abresch R T, Nicorici A, Elfring G L, Atkinson L, Reha A, Hirawat S, Miller L L. The 6-minute walk test as a new outcome measure in Duchenne muscular dystrophy. Muscle Nerve. 2010 April; 41(4):500-10
12. Scott E, Eagle M, Mayhew A, Freeman J, Main M, Sheehan J, Manzur A, Muntoni F; North Star Clinical Network for Paediatric Neuromuscular Disease. Development of a functional assessment scale for ambulatory boys with Duchenne muscular dystrophy. Physiother Res Int. 2012 June; 17(2):101-9. doi: 10.1002/pri.520.
13. Stephenson, K. A., Rae, M. G., & O'Malley, D. (2020). Interleukin-6: A neuro-active cytokine contributing to cognitive impairment in Duchenne muscular dystrophy?. Cytokine, 133, 155134. https://doi.org/10.1016/j.cyto.2020.155134
14. Mammen A L, Sartorelli V. IL-6 Blockade as a Therapeutic Approach for Duchenne Muscular Dystrophy. EBioMedicine. 2015 Mar. 28; 2(4):274-5. doi: 10.1016/j.ebiom.2015.03.018. PMID: 26137566; PMCID: PMC4486189.
15. Mann C J, Perdiguero E, Kharraz Y, Aguilar S, Pessina P, Serrano A L, Munoz-Canoves P. Aberrant repair and fibrosis development in skeletal muscle. Skelet Muscle. 2011 May 4; 1(1):21. doi: 10.1186/2044-5040-1-21.
16. Ceco E, McNally E M. Modifying muscular dystrophy through transforming growth factor-β. FEBS J. 2013 September; 280(17):4198-209. doi: 10.1111/febs.12266. Epub 2013 Apr. 24. PMID: 23551962; PMCID: PMC3731412.
17. Shimizu-Motohashi Y, Komaki H, Motohashi N, Takeda S, Yokota T, Aoki Y. Restoring Dystrophin Expression in Duchenne Muscular Dystrophy: Current Status of Therapeutic Approaches. J Pers Med. 2019
18. Mendell, J. R., Rodino-Klapac, L., Sahenk, Z., Malik, V., Kaspar, B. K., Walker, C. M., & Clark, K. R. (2012). Gene therapy for muscular dystrophy: lessons learned and path forward. Neuroscience letters, 527(2), 90-99.
19. Wells D. J. (2019). What is the level of dystrophin expression required for effective therapy of Duchenne muscular dystrophy?. Journal of muscle research and cell motility, 40(2), 141-150.
20. Awano, H., Matsumoto, M., Nagai, M., Shirakawa, T., Maruyama, N., lijima, K., Nabeshima, Y. I., & Matsuo, M. (2018). Diagnostic and clinical significance of the titin fragment in urine of Duchenne muscular dystrophy patients. Clinica chimica acta; international journal of clinical chemistry, 476, 111-116.
21. Garrood, P., Eagle, M., Jardine, P. E., Bushby, K., & Straub, V. (2008). Myoglobinuria in boys with Duchenne muscular dystrophy on corticosteroid therapy. Neuromuscular disorders: NMD, 18(1), 71-73.
22. Raimondo T_{M}, Mooney D J. Anti-inflammatory nanoparticles significantly improve muscle function in a murine model of advanced muscular dystrophy. Sci Adv. 2021 Jun. 23; 7(26): eabh3693.
23. Thomas G D, Ye J, De Nardi C, Monopoli A, Ongini E, Victor R G. Treatment with a nitric oxide-donating NSAID alleviates functional muscle ischemia in the mouse model of Duchenne muscular dystrophy. PLoS One. 2012; 7(11): e49350.
24. Ryder S, Leadley R M, Armstrong N, Westwood M, de Kock S, Butt T, Jain M, Kleijnen J. The burden, epidemiology, costs and treatment for Duchenne muscular dystrophy: an evidence review. Orphanet J Rare Dis. 2017 Apr. 26; 12(1):79
25. Kieny P, Chollet S, Delalande P, Le Fort M, Magot A, Pereon Y, Perrouin Verbe B. Evolution of life expectancy of patients with Duchenne muscular dystrophy at AFM Yolaine de Kepper centre between 1981 and 2011. Ann Phys Rehabil Med. 2013 September; 56(6):443-54.
26. Duan D. Systemic AAV Micro-dystrophin Gene Therapy for Duchenne Muscular Dystrophy. Mol Ther. 2018 Oct. 3; 26(10):2337-2356.
27. Echevarria L, Aupy P, Goyenvalle A. Exon-skipping advances for Duchenne muscular dystrophy. Hum Mol Genet. 2018 Aug. 1; 27(R2): R163-R172.
28. Dzierlega K, Yokota T. Optimization of antisense-mediated exon skipping for Duchenne muscular dystrophy. Gene Ther. 2020 September; 27(9):407-416. doi: 10.1038/s41434-020-0156-6.
29. Olson E N. Toward the correction of muscular dystrophy by gene editing. Proc Natl Acad Sci USA. 2021 Jun. 1; 118(22): e2004840117.
30. Kourakis S, Timpani C A, Campelj D G, Hafner P, Gueven N, Fischer D, Rybalka E. Standard of care versus new-wave corticosteroids in the treatment of Duchenne muscular dystrophy: Can we do better? Orphanet J Rare Dis. 2021 Mar. 4; 16(1):117. doi: 10.1186/s13023-021-01758-9.
31. Boziki M K, Kesidou E, Theotokis P, Mentis A A, Karafoulidou E, Melnikov M, Sviridova A, Rogovski V, Boyko A, Grigoriadis N. Microbiome in Multiple Sclerosis; Where Are We, What We Know and Do Not Know. Brain Sci. 2020 Apr. 14; 10(4):234. doi: 10.3390/brainsci10040234. PMID: 32295236; PMCID: PMC7226078.
32. Raghavan K, Dedeepiya V D, Yamamoto N, Ikewaki N, Sonoda T, Kurosawa G, Iwasaki M, Kandaswamy R, Senthilkumar R, Preethy S, Abraham S J K. Beneficial reconstitution of gut microbiota and control of alpha-synuclein and curli-amyloids-producing enterobacteria, by beta 1,3-1,6 glucans in a clinical pilot study of autism and potentials in neurodegenerative diseases. medRxiv 2021.10.26.21265505; doi: 10.1101/2021.10.26.21265505
33. Ito K, Kimura S, Ozasa S, Matsukura M, Ikezawa M, Yoshioka K, Ueno H, Suzuki M, Araki K, Yamamura K, Miwa T, Dickson G, Thomas G D, Miike T. Smooth muscle-specific dystrophin expression improves aberrant vasoregulation in mdx mice. Hum Mol Genet. 2006 Jul. 15; 15(14):2266-75.
34. Gajendran N. The root cause of Duchenne muscular dystrophy is the lack of dystrophin in smooth muscle of blood vessels rather than in skeletal muscle per se [version 2; peer review: 3 not approved]. F1000Research 2018, 7:1321. https://doi.org/10.12688/f1000research.15889.2
35. Verhaart I E C, Johnson A, Thakrar S, Vroom E, De Angelis F, Muntoni F, Aartsma-Rus A M, Niks E H. Muscle biopsies in clinical trials for Duchenne muscular dystrophy—Patients' and caregivers' perspective. Neuromuscul Disord. 2019 August; 29(8):576-584
36. Kodippili K, Thorne P K, Laughlin M H, Duan D. Dystrophin deficiency impairs vascular structure and function in the canine model of Duchenne muscular dystrophy. J Pathol. 2021 August; 254(5):589-605.
37. Augier N, Boucraut J, Leger J, Anoal M, Nicholson L V, Voelkel M A, Leger J J, Pellissier J F. A homologue of dystrophin is expressed at the blood vessel membrane of DMD and BMD patients: immunological evidence. J Neurol Sci. 1992 February; 107(2):233-8.
38. Rauch U, Shami A, Zhang F, Carmignac V, Durbeej M, Hultgardh-Nilsson A. Increased neointimal thickening in dystrophin-deficient mdx mice. PLoS One. 2012; 7(1): e29904.
39. Guenio et al. The Female mdx Mouse: An Unexpected Vascular Story.Journal of Neurology & Neuromedicine 2016. https://doi.org/10.29245/2572.942X/2016/2.1014
40. Podkalicka P, Mucha O, Dulak J, Loboda A. Targeting angiogenesis in Duchenne muscular dystrophy. Cell Mol Life Sci. 2019 April; 76(8):1507-1528. doi: 10.1007/s00018-019-03006-7. Epub 2019 Feb. 15. PMID: 30770952; PMCID: PMC6439152.
41. Manickam R, Duszka K, Wahli W. PPARs and Microbiota in Skeletal Muscle Health and Wasting. Int J Mol Sci. 2020 Oct. 29; 21(21):8056.
42. Raghavan K, Dedeepiya V D, Srinivasan S, Pushkala S, Subramanian S, Ikewaki N, Iwasaki M, Senthilkumar R, Preethy S, Abraham S. Disease-modifying immune-modulatory effects of the N-163 strain of Aureobasidium pullulans-produced 1,3-1,6 Beta glucans in young boys with Duchenne muscular dystrophy: Results of an open-label, prospective, randomized, comparative clinical study. medRxiv 2021.12.13.21267706; doi: 10.1101/2021.12.13.21267706
43. Danielle A. Debruin, Nicola Andreacchio, Erik D. Hanson, Cara A. Timpani, Emma Rybalka, and Alan Hayes, The Effect of Vitamin D Supplementation on Skeletal Muscle in the mdx Mouse Model of Duchenne Muscular Dystrophy, Sports (Basel), 2019 May; 7 (5) 96
44. Gang Han, Caorui Lin, Hanhan Ning, Xianjun Gao, and HaiFang Yin, Long-Term Morpholino Oligomers in Hexose Elicit Long-Lasting Therapeutic Improvements in mdx Mice, Molecular Therapy: Nucleic Acids Vol. 12 Sep. 2018
45. SANDRA MURPHY, PAUL DOWLING, MARGIT ZWEYER, MICHAEL HENRY, PAULA MELEADY, RUSTAM R. MUNDEGAR, DIETER SWANDULLA and KAY OHLENDIECK, Proteomic profiling of mdx-4cv serum reveals highly elevated levels of the inflammation-induced plasma marker haptoglobin in muscular dystrophy, DOI: 10.3892/ijmm.2017.2952
46. Eiji Wada, Takayuki Hamano, Isao Matsui, Mizuko Yoshida, Yukiko K. Hayashi and Ryoichi Matsuda, Renal involvement in the pathogenesis of mineral and bone disorder in dystrophin-deficient mdx mouse, The Journal of Physiological Sciences volume 69, pages 661-671 (2019)
47. Esther Lutgens, Marion Gijbels, Marjan Smook, Peter Heeringa, Philip Gotwals, Victor E. Koteliansky and Mat J. A. P. Daemen, Transforming Growth Factor-βMediates Balance Between Inflammation and Fibrosis During Plaque Progression, Arteriosclerosis, Thrombosis, and Vascular Biology. 2002; 22:975-982
48. Merryl Rodrigues, Yusuke Echigoya, Rika Maruyama, Kenji Rowel Q. Lim, So-ichiro Fukada & Toshifumi Yokota, Impaired regenerative capacity and lower revertant fibre expansion in dystrophin-deficient mdx muscles on DBA/2 background, Scientific Reports volume 6, Article number: 38371 (2016)
49. Werner Klingler, Karin Jurkat-Rott, Frank Lehmann-Horn and Robert Schleip, The role of fibrosis in Duchenne muscular dystrophy, Acta Myologica, 2012; XXXI: p. 184-195
50. Patricia Pinol-Jurado, Xavier Suarez-Calvet, Esther Fernandez-Simon, Eduard Gallardo, Natalia de la Oliva, Anna Martinez-Muriana, Pedro Gomez-Galvez, Luis M Escudero, Maria Perez-Peiro, Lutz Wollin, Noemi de Luna, Xavier Navarro, Isabel Ilila, Jordi Diaz-Manera, Nintedanib decreases muscle fibrosis and improves muscle function in a murine model of dystrophinopathy, DOI: 10.1038/s41419-018-0792-6
51. Rebeca Martin, Sylvie S. Miquel, Leandro Benevides, Chantal Bridonneau, Veronique Robert, Sylvie Hudault, Florian Chain, Olivier Berteau, Vasco Azevedo Jean-Marc Chatel, Harry Sokol, Luis Bermudez-Humaran, Muriel Thomas, Philippe Langella, Functional characterization of novel Faecalibacterium prausnitzii strains isolated from healthy volunteers: a step forward in the use of F. prausnitzii as a next-generation probiotic, Frontiers in Microbiology, June 2017, Volume 8, Article 1226
52. Florence Gizard, Anne Fernandez, and Filipe De Vadder, Interactions between gut microbiota and skeletal muscle, doi: 10.1177/1178638820980490
53. Valerio Baldelli, Franco Scaldaferri, Lorenza Putignani and Federica Del Chierico, The Role of Enterobacteriaceae in Gut Microbiota Dysbiosis in Inflammatory Bowel Diseases, Microorganisms 2021, 9(4), 697
54. Zohreh Tamanai-Shacoori, Imen Smida, Latifa Bousarghin, Olivier Loreal, Vincent Meuric, Shao Bing Fong, Martine Bonnaure-Mallet, Anne Jolivet-Gougeon, Roseburia spp.: a marker of health?, Future Microbiol. 2017 February; 12: 157-170
55. Shunjie Bai1, Huili Bai, Detao Li1, Qi Zhong, Jing Xie and Jian-jun Chen, Gut Microbiota-Related Inflammation Factors as a Potential Biomarker for Diagnosing Major Depressive Disorder, Front. Cell. Infect. Microbiol., 15 Mar. 2022
56. Anna Heintz-Buschart, Urvashi Pandey, Tamara Wicke, Friederike Sixel-Doring, Annette Janzen, Elisabeth Sittig-Wiegand, Claudia Trenkwalder, Wolfgang H Oertel, Brit Mollenhauer, Paul Wilmes, The nasal and gut microbiome in Parkinson's disease and idiopathic rapid eye movement sleep behavior disorder, Mov Disord. 2018 January; 33(1):88-98
57. Satvik Mareedu, Emily D. Million, Dongsheng Duan, and Gopal J. Babu, Abnormal Calcium Handling in Duchenne Muscular Dystrophy: Mechanisms and Potential Therapies, Front. Physiol., 9 Apr. 2021 Sec. Striated Muscle Physiology
58. Twee T Do, et al., Muscular Dystrophy Workup, Medscape, Updated: Jul. 1, 2021
59. Yuling Zhu, Huili Zhang, Yiming Sun, Yaqin Li, Langhui Deng, Xingxuan Wen, Huaqiao Wang, Cheng Zhang, Serum Enzyme Profiles Differentiate Five Types of Muscular Dystrophy, Dis Markers. 2015; 2015:543282
60. Andrew L. Mammen and Vittorio Sartorelli, IL-6 Blockade as a Therapeutic Approach for Duchenne Muscular Dystrophy, EBioMedicine. 2015 April; 2(4): 274-275
61. Oriana Del Rocio Cruz-Guzman, Maricela Rodriguez-Cruz, Rosa Elena Escobar Cedillo, Systemic Inflammation in Duchenne Muscular Dystrophy: Association with Muscle Function and Nutritional Status, Biomed Res Int. 2015; 2015:891972
62. https://healthmatters.io/understand-blood-test-results/firmicutes
63. Spase Stojanov, Ales Berlec, and Borut Strukelj, The Influence of Probiotics on the Firmicutes/Bacteroidetes Ratio in the Treatment of Obesity and Inflammatory Bowel disease, Microorganisms 2020, 8(11), 1715
64. Ashutosh K Mangalam, Joseph Murray, Microbial monotherapy with Prevotella histicola for patients with multiple sclerosis, Expert Rev Neurother. 2019 January; 19(1):45-53.
65. Brandi L. Cantarel, Emmanuelle Waubant, Christel Chehoud, Justin Kuczynski, Todd Z. DeSantis, Janet Warrington, Arun Venkatesan, Claire M. Fraser, and Ellen M. Mowry, Gut microbiota in MS: possible influence of immunomodulators, J Investig Med. 2015 June; 63(5): 729-734.
66. Harry Sokol, Benedicte Pigneur, Laurie Watterlot, and Philippe Langella, Faecalibacterium prausnitzii is an anti-inflammatory commensal bacterium identified by gut microbiota analysis of Crohn disease patients, PNAS, Oct. 28, 2008 vol. 105, no. 43, 16731-1673667. Lixing Zhou, Mingming Zhang, Yuming Wang, Robert Gregory Dorfman, Hang Liu, Ting Yu, Xiaotian Chen, Dehua Tang, Lei Xu, Yuyao Yin, Yida Pan, Qian Zhou, Yihua Zhou, Chenggong Yu, Faecalibacterium prausnitzii Produces Butyrate to Maintain Th17/Treg Balance and to Ameliorate Colorectal Colitis by Inhibiting Histone Deacetylase 1, Inflamm Bowel Dis. 2018 Aug. 16; 24(9):1926-1940.
68. Rebeca Martin, Sylvie S. Miquel, Leandro Benevides, Chantal Bridonneau, Veronique Robert, Sylvie Hudault, Florian Chain, Olivier Berteau, Vasco Azevedo, Jean-Marc Chatel, Harry Sokol, Luis Bermudez-Humaran, Muriel Thomas, Philippe Langella, Functional characterization of novel Faecalibacterium prausnitzii strains isolated from healthy volunteers: a step forward in the use of F. prausnitzii as a next-generation probiotic, Front. Microbiol., 30 Jun. 2017 Sec. Food Microbiology
69. Shailesh K. Shahi, Samantha N. Freedman, and Ashutosh K. Mangalam, Gut microbiome in multiple sclerosis: The players involved and the roles they play, Gut Microbes. 2017; 8(6): 607-615.
70. Mehdi Toghi, Sara Bitarafan, Hosein Delavar Kasmaei, Soudeh Ghafouri-Fard, Bifidobacteria: A probable missing puzzle piece in the pathogenesis of multiple sclerosis, Multiple Sclerosis and Related Disorders, Volume 36, November 2019, 101378
71. Daiki Takewaki, Wataru Suda, Wakiro Sato, Lena Takayasu, Naveen Kumar, Kimitoshi Kimura, Naoko Kaga, Toshiki Mizuno, Sachiko Miyake, Masahira Hattori, and Takashi Yamamura, Alterations of the gut ecological and functional microenvironment in different stages of multiple sclerosis, 22402-22412, PNAS, Sep. 8, 2020, vol. 117, no. 36
72. Egle Cekanaviciute, Bryan B. Yoo, Tessel F. Runia, Justine W. Debelius, Sneha Singh, Charlotte A. Nelson, Rachel Kanner, Yadira Bencosme, Yun Kyung Lee, Stephen L. Hauser, Elizabeth Crabtree-Hartman, Ilana Katz Sand, Mar Gacias, Yunjiao Zhu, Patrizia Casaccia, Bruce A. C. Cree, Rob Knight, Sarkis K. Mazmanian, and Sergio E. Baranzini, Gut bacteria from multiple sclerosis patients modulate human T cells and exacerbate symptoms in mouse models, PNAS, Oct. 3, 2017, vol. 114, no. 40, 10713-10718
73. Kai Nie, Kejia Ma, Weiwei Luo, Zhaohua Shen, Zhenyu Yang, Mengwei Xiao, Ting Tong, Yuanyuan Yang, and Xiaoyan Wang, Roseburia intestinalis: A Beneficial Gut Organism From the Discoveries in Genus and Species, Front. Cell. Infect. Microbiol., 22 Nov. 2021, Sec. Bacteria and Host

1. A composition for improving, preventing and/or treating muscular dystrophy and/or multiple sclerosis, comprising a beta-glucan.

2. The composition of claim 1, in which the beta-glucan comprises a beta-glucan produced by Aureobasidium pullulans N-163 (NITE P-03377).

3. The composition of claim 1, which is used to improve, prevent and/or treat Duchenne muscular dystrophy (DMD).

4. A method of improving, preventing and/or treating muscular dystrophy and/or multiple sclerosis in a subject, comprising administrating a composition comprising a beta-glucan to the subject in need thereof.

5. The method of claim 4, wherein the beta-glucan comprises a beta-glucan produced by Aureobasidium pullulans N-163 (NITE P-03377).

6. The method of claim 4, wherein muscular dystrophy is Duchenne muscular dystrophy (DMD).
